(19) United States
(12) Patent Application Publication
(10) Pub. No.: US 2005/0059596 A1
(43) Pub. Date: Mar. 17, 2005

(54) Modulating response to genotoxic stress

(75) Inventor: Jay H. Chung, Bethesda, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Government of the U.S.A. as represented by the Secretary of the Dept. of Health & Human Services

(21) Appl. No.: 10/916,083

(22) Filed: Aug. 10, 2004

Related U.S. Application Data
(62) Division of application No. 09/947,258, filed on Sep. 5, 2001, now abandoned.
(60) Provisional application No. 60/230,476, filed on Sep. 6, 2000.

(51) Int. Cl. C12Q 1/68; C12N 15/85

(52) U.S. Cl. 514/12; 435/455

(57) ABSTRACT

Methods are disclosed to modify cellular sensitivity to genotoxic stress by introducing into a cell a biological macromolecule that alters phosphorylation of BRCA1 by Cds1. Such biological macromolecules may be used to enhance genotoxin-induced cell death, or alter genotoxin-induced gene expression. Alternatively, such biological macromolecules may be used to enhance cell survival following exposure to genotoxic agents. Methods are also disclosed for determining exposure to genotoxic stress by assessing the phosphorylation status of BRCA1, for example by using antibody molecules disclosed herein.

### PRIORITY INFORMATION

This application claims priority to U.S. Provisional Application No: 60/230,476, filed Sep. 6, 2000, herein incorporated by reference in its entirety.

### FIELD

This application relates to methods and compositions which can be used to modify cellular sensitivity to genotoxic stress.

### BACKGROUND

Genotoxic stress, defined generally as genotoxin-induced cellular DNA damage, is a major cause of human and veterinary illness. Illnesses attributable to genotoxic stress include aging, cancer, and some forms of heart failure. Genotoxins that induce such cellular DNA damage include ultraviolet light, ionizing radiation, and chemotherapy agents including anthracyclines, cisplatin, and cyclophosphamide.

Upon induction of such DNA damage, a normal cell in an organism activates a number of defensive responses to protect the cell and/or the organism. For example, cell cycle checkpoints may be activated that block DNA duplication (known as the G1/S checkpoint) and cell division (known as the G2/M checkpoint) until the cell can repair the DNA damage. Alternatively, the damage may activate pathways leading to programmed cell death, or apoptosis. Such cell death in response to DNA damage may prevent untoward events such as cancerous transformation of the cell.

Checkpoints maintain order and fidelity in the cell cycle, and thereby play a crucial role in the organism's response to genotoxin-induced cellular DNA damage. In the eukaryotic cell cycle, the G2-M checkpoint operates in G2 phase and blocks entry into mitosis when damaged or unreplicated DNA is detected. The G1/S checkpoint operates at the G1/S boundary, controlling entry into the S phase of DNA replication.

Genetic studies have revealed that these checkpoints require the action of kinases that are activated by genotoxic stress. These kinases include ATM (Ataxia-Telangiecstasia Mutated, so named because it was discovered in cells from patients with the cancer-susceptibility syndrome ataxia-telangiecstasia), and ATR (Ataxia-Telangiecstasia Related). ATM is known to directly phosphorylate the tumor suppressor gene p53, thereby (1) inhibiting cell cycle progression into S phase (by activating expression of p21waf1/cip1, which induces cell cycle block at the G1/S boundary) and (2) promoting apoptosis in DNA-damaged cells. In contrast, ATR likely blocks cell cycle progression in cells that have damaged DNA and/or have failed to correctly replicate their DNA, by activating the G2/M checkpoint.

What is needed are agents and methods that help generally healthy cells to survive genotoxic stress without becoming more prone to cancerous transformation. What is also independently needed are agents and methods that enhance the sensitivity of diseased cells such as cancer cells to genotoxins such as UV radiation, ionizing radiation, and chemotherapeutic agents. Another independent need is reagents and methods for identifying cells that have been exposed to genotoxic stress.

### SUMMARY OF THE DISCLOSURE

The present application discloses methods for modifying cellular sensitivity to genotoxic stress. In one embodiment, the method involves exposing a cell to a biological macromolecule that alters phosphorylation of BRCA1 by Cds1, a stress-activated kinase that mediates cellular responses to genotoxic agents such as ionizing radiation, irradiation, and chemotherapy. Data presented herein demonstrates that phosphorylation of BRCA1 by Cds1 activates a variety of intracellular signaling pathways that affect cell survival. Methods are provided for modulating these intracellular signaling pathways to either enhance or reduce cell survival.

A method of modulating expression from a nucleic acid sequence modulated by BRCA1 in a cell, by introducing into the cell a nucleic acid encoding BRCA1 or a functional fragment or variant thereof, wherein the nucleic acid includes a mutation that encodes amino acid substitution that alters Cds1 phosphorylation of BRCA1, is disclosed.

Also disclosed herein are antibodies specific for BRCA1 peptides including a phosphorylated serine residue at a position corresponding to amino acid residue 988 of a human BRCA1 polypeptide, and methods of making such antibodies.

Further disclosed herein is a method of determining exposure to genotoxic stress by determining whether S988 of BRCA1 in a cell is phosphorylated, for example by using the antibodies disclosed herein.

### SEQUENCE LISTING

The nucleic and amino acid sequences in the accompanying sequence listing are shown using standard letter abbreviations for nucleotides, and three letter code for amino acids. Only one strand of each nucleic acid sequence is shown, but the complementary strand is understood as included by any reference to the displayed strand.

SEQ ID NO: 1 shows an amino acid sequence used to generated a polyclonal antibody specific for phosphorylated Ser 988 of BRCA1.

SEQ ID NOS: 2 and 3 show nucleic acid probes specific for an estrogen response element (ERE).

SEQ ID NO: 4 shows an amino acid sequence which corresponds to residues 978-993 of human BRCA1, with ala substituted for ser at residue 988.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a protein” includes a plurality of such proteins and reference to “the antibody” includes reference to one or more antibodies and equivalents thereof known to those skilled in the art, and so forth.

Unless explained otherwise, all technical and scientific terms used herein have the same meaning as commonly understood to one of ordinary skill in the art to which this disclosure belongs. Definitions of common terms in molecular biology may be found in Benjamin Lewin, Genes VII, published by Oxford University Press, 1999; Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Although exemplary methods and materials are described below, similar or equivalent methods and materials to those described herein can be used to practice the present disclosure. The materials, methods, and examples are illustrative only and not intended to be limiting.

Biological Macromolecule: A term which includes, but is not limited to: polypeptides, polynucleotides, lipids, carbohydrates and other biological molecules having a molecular weight of about 500 Daltons or greater. The term also includes complex or hybrid molecules such as lipoproteins, nucleoproteins, glycoproteins, etc. Such biological macromolecules are normally synthesized intracellularly from precursor molecules. See Stryer, Biochemistry 3rd. Ed., 1988; Alberts et al., Eds., Molecular Biology of the Cell, 3rd Ed. 1994.

BRCA1 cDNA: As used herein, a BRCA1 cDNA-includes BRCA1 cDNAs from any organism, such as mammals, for example human and murine cDNAs. BRCA1 cDNA can be derived by reverse transcription from the mRNA encoded by a BRCA1 gene and lacks internal non-coding segments and transcription regulatory sequences present in a BRCA1 gene. Includes sequence variants, fragments, polymorphisms, mutants and fusions thereof.

BRCA1 gene: A gene which encodes a BRCA1 protein from any organism, such as a mammalian BRCA1 protein, such as a human BRCA1 protein, or a murine BRCA1 protein. A BRCA1 gene includes the various sequence polymorphisms and allelic variants that exist within and between species.

BRCA1 is a tumor suppressor gene, which in some embodiments is mutated in families with inherited breast and ovarian cancer (U.S. Pat. No. 5,709,999; Miki et al., Science 266:66-71, 1994). BRCA1 may play a role in DNA damage repair (Gowen et al., Science 281:1009-12, 1998; Shen et al., Oncogene 17:3115-24, 1998; Abbott et al., J. Biol. Chem. 274:18808-12, 1999; Monyhan et al., Mol. Cell 4:511-8, 1999), cell cycle checkpoint regulation (Somasundaram et al., Nature 389:187-90, 1997; Larson et al., Cancer Res. 57:3351-5, 1997; Xu et al., Mol. Cell 3:389-95, 1999), and transcription regulation, although its exact biochemical function remains to be established.

BRCA1 protein: A protein encoded by a BRCA1 gene or cDNA from any organism. In one embodiment, a BRCA1 protein includes mammalian BRCA1 proteins, such as a human BRCA1 protein. This description includes natural allelic variants in the disclosed sequences, as well as the protein of any species, and variant, fragment, or fusion peptides which retain BRCA1 functional activity. BRCA1 is normally located in nuclear foci, but upon DNA damage it becomes hyperphosphorylated and disperses (Scully et al., Cell 90:425-35, 1997; Thomas et al., Cell Growth Differ. 8:801-9, 1997).

BRCA1 phosphorylation by Cds1 is said to be altered by a biological macromolecule if the biological molecule increases or decreases phosphorylation of at least one BRCA1 amino acid residue by Cds1, such as S988 of human BRCA1 (and the corresponding serine in other organisms; see Table 1). Assays which can be used to determine a relative amount of phosphorylation are disclosed herein, for example, the methods disclosed in Examples 1 and 2 (in vitro kinase assays), Example 4 (Western blotting), Example 6 (immunofluorescence), Example 7 (co-immunoprecipitation), Example 8 (survival assays) and Example 9 (luciferase assays).

In one embodiment, a reduction or decrease in phosphorylation is a reduction by at least 2-fold, for example at least 5-fold, for example at least 10-fold, for example at least 20-fold, relative to Cds1 phosphorylation of BRCA1 in unirradiated cells. In another embodiment, an increase or enhancement of phosphorylation is an increase by at least 2-fold, for example at least 5-fold, for example at least 10-fold, for example at least 20-fold, relative to Cds1 phosphorylation of BRCA1 in unirradiated cells.

Cancer: Malignant neoplasm that has undergone characteristic anaplasia with loss of differentiation, increase rate of growth, invasion of surrounding tissue, and is capable of metastasis.

Chemotherapy: In cancer treatment, chemotherapy refers to the administration of one or a combination of compounds to kill or slow the reproduction of rapidly multiplying cells. In rheumatology, chemotherapy is often designed to decrease the abnormal behavior of cells, rather than kill cells. The amount of chemotherapeutic agent used for rheumatic or autoimmune conditions are usually lower than the doses used for cancer treatment. Chemotherapuetic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol.

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Cds1: A stress-activated intracellular kinase originally identified in the fission yeast S. pombe (GenBank Accession No. AA773443). Mammalian and human homologs are described in Matsuoka et al., Science 282:1893-7, 1998; and Brown et al., PNAS 96:3745-50, 1999. Examples of Cds1 sequences that can be used to practice the methods disclosed herein include, but is not limited to GenBank Accession No. AF096279. This description includes natural Cds1 allelic variants, as well as the protein of any species, and variant, fragment, or fusion peptides which retain Cds1 functional activity.

Chemical synthesis: An artificial means by which one can make a protein or peptide. A synthetic protein or peptide is one made by such artificial means.

Comprises: A term that means “including.”

Deoxyribonucleic acid (DNA): A long chain polymer comprising the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide. The term codon is also used for the corresponding (and complementary) sequences of three nucleotides in the mRNA into which the DNA sequence is transcribed.

Deletion: The removal of a sequence of a nucleic acid, such as DNA or RNA, the regions on either side being joined together.

Encode: A polynucleotide encodes a polypeptide if, in its native state or when manipulated by methods known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

Estrogen receptor (ER): A ligand-activated transcription factor that mediates the effects of the steroid hormone 17 beta-estradiol. There are two known subtypes, ER alpha (herein ERa) and ER beta. See Enmark et al., J. Int. Med. 246:133-8, 1999; Dechering et al., Curr. Med. Chem. 7:561-76, 2000.

Functional fragments and variants of a polypeptide: Includes those fragments and variants that maintain one or more functions of the parent polypeptide. It is recognized that the gene or cDNA encoding a polypeptide may be considerably mutated without materially altering one or more the polypeptide's functions. First, the genetic code is well-known to be degenerate, and thus different codons encode the same amino acids. Second, even where an amino acid substitution is introduced, the mutation may be conservative and have no material impact on the essential functions of the protein. See Stryer, Biochemistry 3rd Ed., (c) 1988. Third, part of a polypeptide chain may be deleted without impairing or eliminating all of its functions. Fourth, insertions or additions may be made in the polypeptide chain, for example, adding epitope tags, without impairing or eliminating its functions.

Other modifications that can be made without materially impairing one or more functions of a polypeptide include, for example, in vivo or in vitro chemical and biochemical modifications or which incorporate unusual amino acids. Such modifications include, for example, acetylation, carboxylation, phosphorylation, glycosylation, ubiquination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands.

For example, hCds1 is a kinase involved in several intracellular signal transduction pathways, many of which are activated by exposure to genotoxic agents. It has serine-threonine kinase activity towards numerous substrates, for example Cdc25c, BRCA1, and itself (autophosphorylation). To accomplish these phosphorylations, it binds ATP, binds to the substrate polypeptide, catalyzes insertion of a phosphate onto the side-chain of serine in the polypeptide, releases ADP, etc. In addition, it is itself a substrate for other kinases, such as AT proteins (ATM and ATR); accordingly, it may bind to these kinases, become phosphorylated by them. A functional fragment or variant of hCds1 is one that maintains at least one of its functions.

For example, hCds1 harboring a lys to arg mutation at position 249 (hCds1 [K249R]) is “kinase-dead,” that is, it lacks the ability to catalyze insertion of a phosphate onto many hCds1 substrates. However, it maintains the ability to bind many hCds1 substrates, for example BRCA1 and Cdc25c, and therefore is defined as a “functional fragment or variant” of hCds1.

Functional fragments and variants also include those in which a function is enhanced. For example, hCds1 mutations may produce enhanced kinase activity, kinase activity in the absence of stimulation by genotoxic agents, or kinase activity in the absence of phosphorylation by AT proteins.

Similarly, BRCA1 may have many intracellular functions in DNA damage repair, cell cycle checkpoint regulation, and regulation of transcription. To accomplish these functions, it may interact with other intracellular macromolecules, and in some instances modify or be modified by these macromolecules. For example, BRCA1 may be phosphorylated by kinases. A functional fragment or variant of BRCA1 is one that maintains one or more functions of BRCA1.

A fragment of BRCA1 that binds to hCds1 is a “functional fragment or variant” of BRCA1, even though it is so substantially truncated, extended, or otherwise mutated as to seriously impair or abolish its other functions (for example, its ability to be phosphorylated by a kinase for which it is a good substrate in the absence of mutation). For example, a fragment of BRCA1 comprising amino acid residues 758-1064, with ser 988 mutated to ala, and with a hemagglutinin epitope tag attached to residue 758, nevertheless at least maintains ability to bind to hCds1. Therefore, it is defined as a “functional fragment or variant” of BRCA1.

The term also includes fragments and variants of BRCA1 in which one or more of its functions are enhanced. Such a BRCA1 fragment or variant may exhibit enhanced binding affinity for one or more macromolecules, for example enhanced affinity for ERa or hCds1; or enhanced ability to stimulate or inhibit transcription of a gene.

Fusion proteins: The production of a protein can be accomplished in a variety of ways (for example see EXAMPLES 17 and 19). DNA sequences which encode for a protein or fusion protein, or a fragment or variant of a protein can be engineered to allow the protein to be expressed in eukaryotic cells or organisms, bacteria, insects, and/or plants. To obtain expression, the DNA sequence can be altered and operably linked to other regulatory sequences. The final product, which contains the regulatory sequences and the therapeutic protein, is referred to as a vector. This vector can be introduced into eukaryotic, bacteria, insect, and/or plant cells. Once inside the cell the vector allows the protein to be produced.

A fusion antigen comprising a protein, such as BRCA1 (or variants, polymorphisms, mutants, or fragments thereof) linked to other amino acid sequences that do not inhibit a desired activity of the protein. In one embodiment, the other amino acid sequences are no more than 10, 20, 30, or 50 amino acid residues in length.

One of ordinary skill in the art will appreciate that the DNA can be altered in numerous ways without affecting the biological activity of the encoded protein. For example, PCR can be used to produce variations in the DNA sequence which encodes an antigen. Such variants can be variants optimized for codon preference in a host cell used to express the protein, or other sequence changes that facilitate expression.

GADD45: A DNA damage and repair related gene in mammalian cells, the expression of which is induced by irradiation and other forms of genotoxic stress. For example, see Genbank accession No: XM_{—}040594. Fomace et al., Proc. Natl. Acad. Sci. USA 85:8800-4, 1988; Carrier et al., J. Biol. Chem. 269:32672-7, 1994; Harkin et al., Cell 97:575-86, 1999.

Genotoxic stress: Genotoxin-induced cellular DNA damage. Cellular responses to genotoxic stress include DNA damage, changes in the cell cycle, apoptosis, and cell death. Examples of agents which induce genotoxic stress include, but are not limited to: ionizing radiation, ultraviolet radiation, and chemotherapeutic agents (such as all those described in Slapak and Kufe, Principles of Cancer Therapy, Ch. 86 in Harrison's Principles of Internal Medicine, 14^{th} Ed. 1998).

An agent is said to modify sensitivity to genotoxic stress if the agent increases or decreases sensitivity to genotoxic stress.

For example, biological macromolecules that exhibit one or more of the following characteristics will, upon administration to a cell, such as a tumor cell, enhance the sensitivity of the cell to genotoxic agents: reduce the ability of BRCA1 to disperse from nuclear foci using the methods described in Example 6, impair enhancement of p21 -mediated expression following irradiation according to Example 9, and/or persistent complex formation with CtIP following irradiation according to Example 10.

Isolated: An isolated biological component (such as a nucleic acid, protein or organelle) has been substantially separated away from other biological components in the cell of the organism in which the component naturally occurs, i.e., other chromosomal and extra-chromosomal DNA and RNA, proteins and organelles. Nucleic acids and proteins that have been isolated include nucleic acids and proteins purified by standard purification methods. The term also embraces nucleic acids and proteins prepared by recombinant expression in a host cell as well as chemically synthesized nucleic acids and proteins.

Mammal: This term includes both human and non-human mammals. Similarly, the terms patient, subject, and individual include both human and veterinary subjects. Examples of mammals include, but are not limited to: humans, pigs, cows, goats, cats, dogs, rabbits and mice.

Modulate expression: Increase or decrease expression of a nucleic acid and/or protein, relative to the wild-type level of expression observed in normal, non-tumor cells.

Neoplasm: Abnormal growth of cells

Oligonucleotide: A linear polynucleotide sequence of up to about 200 nucleotide bases in length, for example a polynucleotide (such as DNA or RNA) which is at least about 6 nucleotides, for example at least 10, 15, 50, 100 or 200 nucleotides long.

Open reading frame (ORF): A series of nucleotide triplets (codons) coding for amino acids without any internal termination codons. These sequences are usually translatable into a peptide.

Operably linked: A first nucleic acid sequence is operably linked to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

p21waf1/cip1: An intracellular protein that plays a role in regulating cell growth and the cell response to DNA damage. The cDNA encoding p21waf1/cip1 has been described by Xiong et al., Nature 366:701-5, 1993, incorporated herein by reference. The primary targets of p21waf1/cip1 are the cdk-cyclins which regulate the progression of eukaryotic cells through the cell cycle, and proliferating cell nuclear antigen (PCNA), an accessory protein of DNA polymerase delta. p21 forms complexes with a class of cdk-cyclins to inhibit their kinase activity and with PCNA to inhibit DNA synthesis. Transcriptional control of p21 by factors other than p53 is critical for growth arrest and for cell differentiation in many instances. See U.S. Pat. No. 6,053,300; Gorospe et al., Gene Expression 7:377-85, 1999; Chen et al., J. Cell. Phys. 181:385-92, 1999; Boulaire et al., Pathologie Biologie 48:190-202, 2000.

PCAF (p300/CBP associated factor): An intracellular histone acteyltranferase that functions as a coactivator for several transcription factors, including nuclear hormone receptors and p53. It participates in transcription by forming an activation complex and by promoting histone acetylation. For example, see GenBank Accession No: XM_{—}010914; PCT WO 98/03652A2; Ogryzko et al., Cell 87:953-9, 1996; Schlitz et al., Biochim. Biophys. Acta 1470:M37-53, 2000.

Probes and primers: Nucleic acid probes and primers can be readily prepared based on the nucleic acid molecules provided herein. A probe comprises an isolated nucleic acid attached to a detectable label or reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., in Sambrook et al. (In Molecular Cloning: A Laboratory Manual, CSHL, New York, 1989) and Ausubel et al. (In Current Protocols in Molecular Biology, Greene Publ. Assoc. and Wiley-Intersciences, 1992).

Primers are short nucleic acid molecules, for example, oligonucleotides 10 nucleotides or more in length. Longer DNA oligonucleotides can be about 15, 17, 20, 23 or 25 nucleotides long. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (In Molecular Cloning: A Laboratory Manual, CSHL, New York, 1989), Ausubel et al. (In Current Protocols in Molecular Biology, Greene Publ. Assoc. and Wiley-Intersciences, 1992), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, for example, a primer comprising 30 consecutive nucleotides of an hCds1 encoding nucleotide will anneal to a target sequence, such as another hCds1 gene homolog from the gene family contained within a human genomic DNA library, with a higher specificity than a corresponding primer of only 15 nucleotides. Thus, to obtain greater specificity, probes and primers can be selected that comprise at least 17, 20, 23, 25, 30, 35, 40, 45, 50 or more consecutive nucleotides of an hCds1 nucleotide sequence.

The disclosure thus includes isolated nucleic acid molecules comprising specified lengths of a cDNA sequence. Such molecules may comprise at least 17, 20, 23, 25, 30, 35, 40, 45 or 50 consecutive nucleotides of a sequences, and may be obtained from any region of the disclosed sequences. For example, an hCds1 cDNA, ORF, coding sequence, or gene sequence can be apportioned into halves or quarters based on sequence length, and the isolated nucleic acid molecules (e.g., oligonucleotides) derived from the first or second halves of the molecules, or any of the four quarters. In addition, a nucleic acid sequence, such as a cDNA, can be divided into smaller regions, e.g. about eighths, sixteenths, twentieths, fiftieths and so forth, with similar effect.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Protein: A biological molecule comprised of amino acids. In one embodiment, a protein is expressed by a gene and in another embodiment, is chemically synthesized.

Purified: The term “purified” does not require absolute purity; rather, it is intended as a relative term. Thus, for example, a purified protein or nucleic acid preparation is one in which the protein or nucleic acid referred to is more pure than the protein or nucleic acid in its natural environment within a cell. For example, a preparation of a protein is purified if the protein represents at least 50%, for example at least 70%, of the total protein content of the preparation. Methods for purification of proteins and nucleic acids are well known in the art, for example as disclosed in Sambrook et al. (Molecular Cloning: A Laboratory Manual, Cold Spring Harbor, New York, 1989, Ch. 17).

Recombinant: A recombinant nucleic acid is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acids, e.g., by genetic engineering techniques.

Sample: Biological samples containing genomic DNA, cDNA, RNA, or protein obtained from the cells of a subject, such as those present in peripheral blood, urine, saliva, tissue biopsy, surgical specimen, fine needle aspriates, amniocentesis samples, autopsy material and others known in the art.

Sequence identity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, 75%, 80%, 85%, 90%, 95%, or even 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

Protein homologs are typically characterized by possession of at least 70%, such as at least 75%, 80%, 85%, 90%, 95% or even 98% sequence identity, counted over the full-length alignment with the amino acid sequence using the NCBI Basic Blast 2.0, gapped blastp with databases such as the nr or swissprot database. Queries searched with the blastn program are filtered with DUST (Hancock and Armstrong, 1994, Comput. AppL Biosci. 10:67-70). Other programs use SEG.

One of skill in the art will appreciate that these sequence identity ranges are provided for guidance only; it is possible that strongly significant homologs could be obtained that fall outside the ranges provided. Provided herein are the peptide homologs described above, as well as nucleic acid molecules that encode such homologs.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous peptides can, for example, possess at least 75%, 80%, 90%, 95%, 98%, or 99% sequence identity determined by this method. When less than the entire sequence is being compared for sequence identity, homologs can, for example, possess at least 75%, 85% 90%, 95%, 98% or 99% sequence identity over short windows of 10-20 amino acids. Methods for determining sequence identity over such short windows can be found at the NCBI web site. One of skill in the art will appreciate that these sequence identity ranges are provided for guidance only; it is possible that significant homologs or other variants can be obtained that fall outside the ranges provided.

Specific binding agent: An agent that binds substantially only to a defined target. A BRCA1 specific binding agent binds substantially only a BRCA1 protein. The term “anti-BRCA1 antibodies” encompasses monoclonal and polyclonal antibodies specific for a BRCA1 protein i.e., which bind substantially only to a BRCA1 protein when assessed using the methods described below, as well as immunologically effective portions (“fragments”) thereof. Antibodies disclosed herein can be polyclonal antibodies, monoclonal antibodies (mAb) (or immunologically effective portions thereof) and humanized mAbs (or immunologically effective portions thereof). Immunologically effective portions of mAbs include Fab, Fab′, F(ab′)₂ Fabc, Fv portions as well as any other agent capable of specifically binding to a BRCA1 protein (or the other disclosed proteins). Antibodies can also be produced using standard procedures as described in EXAMPLES 4 and 16, and as described in Harlow and Lane (Antibodies: A Laboratory Manual. 1988).

The determination that a particular agent binds substantially only to a BRCA1 protein can be made using or adapting routine procedures. For example, western blotting can be used to determine that a specific binding agent, such as a mAb, binds substantially only to the protein (Harlow and Lane, Antibodies: A Laboratory Manual. 1988).

Subject: Living multicellular vertebrate organisms, a category which includes, both human and veterinary subjects for example, mammals, rodents, and birds.

Therapeutically Effective Amount: An amount sufficient to achieve a desired biological effect, for example an amount that is effective to increase or decrease (such as inhibit) sensitivity to genotoxic stress and/or expression of a nucleic acid sequence modulated by BRCA1.

In particular examples, it is a concentration of a biological macromolecule, such as a Cds1 or BRCA1 protein or nucleic acid, effective to increase or decrease the sensitivity to genotoxic stress in a cell, such as a cell in a subject to whom it is administered.

In one example, it is an amount of a biological macromolecule, such as a BRCA1 polypeptide including an S988 mutation, such as S988A, or a kinase-dead hCds1 mutant, which increases or enhances the sensitivity of a cell, such as a cancerous cell in a subject, to genotoxic agents, by more than a desired amount.

In other examples, it is an amount of a biological macromolecule, such as a BRCA1 polypeptide including an S988 mutation, such as S988E, effective to decrease or reduce sensitivity of a cell to genotoxic stress by more than a desired amount, such as a normal cell in a subject undergoing genotoxic therapy, to reduce the impact of such therapy.

In one embodiment, the therapeutically effective amount also includes a quantity of a Cds1 and/or BRCA1 protein (including variant, mutant, fragment, or fusion peptides) sufficient to achieve a desired effect in a subject being treated. In another or additional embodiment, the therapeutically effective amount also includes a quantity of Cds1 and/or BRCA1 nucleic acid (including sequence variants, fragments, polymorphisms, mutants and fusions thereof) sufficient to achieve a desired effect in a subject being treated. For instance, these can be an amount necessary to improve signs and/or symptoms a disease such as a skin disease or cancer, for example by modulating sensitivity to genotoxic stress or expression of a nucleic acid sequence modulated by BRCA1.

An effective amount of a biological macromolecule (such as a protein or nucleic acid) can be administered in a single dose, or in several doses, for example daily, during a course of treatment. However, the effective amount of biological macromolecule will be dependent on the source of biological macromolecule administered (i.e. a biological macromolecule isolated from a cellular extract versus a chemically synthesized and purified biological macromolecule), the subject being treated, the severity and type of the condition being treated, and the manner of administration of a biological macromolecule. For example, a therapeutically effective amount of a protein can vary from about 0.01 mg/kg body weight to about 1 g/kg body weight.

The biological macromolecules disclosed herein have equal application in medical and veterinary settings. Therefore, the general term “subject being treated” is understood to include all animals (e.g. humans, apes, dogs, cats, horses, and cows) that require modulation of sensitivity to genotoxic stress and/or expression of a nucleic acid sequence modulated by BRCA1.

Therapeutically effective dose: A dose sufficient to modulate, such as increase or decrease (such as inhibit) sensitivity to genotoxic stress in a cell by altering phosphorylation of BRCA1 by Cds1, resulting in a regression of a pathological condition, or which is capable of relieving signs or symptoms caused by the condition, such as cancer. In another embodiment, it is a dose sufficient to modulate, such as increase or decrease (such as inhibit) expression from a nucleic acid sequence that is modulated by BRCA1 in a cell by introducing into the cell a nucleic acid encoding BRCA1 which includes a mutation that alters Cds1 phosphorylation of BRCA1, resulting in a regression of a pathological condition, or which is capable of relieving signs or symptoms caused by the condition, such as cancer.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: Transformed cells which contain foreign, non-native DNA.

Tumor: A neoplasm. Includes solid and hematological (or liquid) tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrdm's macroglobulinemia, heavy chain disease, myelodysplastic syndrome, and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Vector: A nucleic acid molecule as introduced into a host cell, thereby producing a transformed host cell. A vector can include nucleic acid sequences that permit it to replicate in the host cell, such as an origin of replication. A vector can also include one or more selectable marker genes and other genetic elements known in the art.

Herein disclosed is a method for modifying sensitivity to genotoxic stress, by exposing a cell to a biological macromolecule that alters BRCA1 phosphorylation by Cds1, at one or more BRCA1 amino acid residues that affect a cellular response to genotoxic stress. In one embodiment, exposing the cell to the biological macromolecule is achieved by contacting the cell with the biological macromolecule. In another embodiment, exposing the cell to the biological macromolecule is achieved by administering the biological macromolecule to the cell, for example by introducing the biological macromolecule into cell, such as a mammalian cell. In yet another embodiment, exposing the cell to the biological macromolecule includes administering a therapeutically effective amount of the biological macromolecule to a subject to affect the subject's cellular response to genotoxic stress.

In one embodiment, the biological macromolecule alters phosphorylation by Cds1 of at least one amino acid between human BRCA1 residues 758-1064, for example serine 988 (S988), or homologs of these residues in other organisms (i.e. see Table 1). In another embodiment, the biological macromolecule that alters BRCA1 phosphorylation by Cds1 is a nucleic acid, operably linked to a promoter, that expresses a polypeptide in the cell. In a particular embodiment, the nucleic acid expresses Cds1, or a functional fragment of Cds1. For example, the expressed Cds1 variant may have altered ability to phosphorylate BRCA1, such as a reduced ability to phosphorylate BRCA1, and may include an amino acid substitution at a residue corresponding to lysine 249 of human Cds1, such as an arginine substitution. In other embodiments, the nucleic acid expresses a BRCA1 polypeptide, or a functional fragment or variant of BRCA1 that includes a residue homologous to serine 988 of human BRCA1, for example, a polypeptide including residues homologous to 758-1064 of human BRCA1. In particular embodiments, the expressed BRCA1 polypeptide has an amino acid substitution at a residue homologous to S988 of human BRCA1, for example a nonpolar or hydrophobic substitution; a polar or charged substitution; or an alanine, glutamic acid, or aspartic acid substituted for serine. In other particular embodiments, the expressed BRCA1 polypeptide includes at least 8 residues, for example at least 30 residues, for example at least 100 residues, having at least 70%, 75%, 80%, 85%, 90%, 95%, 98%, or 99% homology to a human BRCA1 amino acid sequence.

In other particular embodiments, the biological macromolecule that modulates BRCA1 phosphorylation by Cds1 is a polypeptide. In one embodiment, the polypeptide is Cds1 or a functional fragment or variant of Cds1 with altered ability, such as reduced ability, to phosphorylate BRCA1, and may include an amino acid substitution such as arginine at a residue corresponding to lysine 249 (K249) of human Cds1. The polypeptide may be a BRCA1 polypeptide that includes a residue homologous to S988 of human BRCA1, for example a polypeptide that includes residues homologous to 758-1064 of human BRCA1. In particular embodiments, the BRCA1 polypeptide may include an amino acid substitution at a residue homologous to S988 of human BRCA1, for example, nonpolar, hydrophobic, polar, or charged substitutions. The substitution may be alanine, glutamic acid, or aspartic acid for serine. In other particular embodiments, the expressed BRCA1 polypeptide includes at least 8 residues, for example at least 30 residues, for example at least 100 residues, having at least 70%, 75%, 80%, 85%, 90%, 95%, 98%, or 99% homology to a human BRCA1 amino acid sequence.

Methods are disclosed for modulating expression from a nucleic acid sequence that is modulated by BRCA1 in a cell, by introducing into the cell a nucleic acid encoding a BRCA1 polypeptide or functional fragments or variants of BRCA1. In one embodiment, introducing into the cell the nucleic acid encoding BRCA1 or a functional fragment or variant thereof including a mutation that encodes an amino acid substitution that alters Cds1 phosphorylation of BRCA1, includes administering a therapeutically effective amount of the nucleic acid encoding BRCA1 or a functional fragment or variant thereof including a mutation that encodes an amino acid substitution that alters Cds1 phosphorylation of BRCA1 to a subject to affect the subject's expression of the nucleic acid sequence that is modulated by BRCA1 in a cell.

In one embodiment, the nucleic acid sequences includes amino acid residues 758-1064 of human BRCA1, or homologs thereof, and may further include an amino acid substitution that alters BRCA1 phosphorylation by Cds1. For example, the substitution may be at a residue homologous to serine 988 of human BRCA1, and may be for example, a nonpolar, hydrophobic, polar, or charged substitution. The substitution may be an alanine, glutamic acid, or aspartic acid substituted for serine. In other particular embodiments, the expressed BRCA1 polypeptide includes at least 8 residues, for example at least 30 residues, for example at least 100 residues, having at least 70%, 75%, 80%, 85%, 90%, 95%, 98%, or 99% homology to a human BRCA1 amino acid sequence. The nucleic acid sequence whose expression is modulated by BRCA1 may be, for example, p21waf1/cip1, GADD45 or sequences whose promoters/enhancers contain at least one estrogen response element (ERE).

In other particular embodiments, the methods for modulating cellular expression of a nucleic acid include expressing a BRCA1 polypeptide with an amino acid substitution that alters binding between BRCA1 and an estrogen receptor (ER), or a transcriptional repressor such as CTIP. In still other particular embodiments, expression of the BRCA1 polypeptide may alter ER interaction with a nuclear receptor coactivator, for example PCAF.

Also disclosed are antibodies, such as a purified antibody, immunologically specific for a BRCA1 polypeptide with a phosphorylated serine at a position homologous to S988 of human BRCA1, and antibody molecules immunologically specific for BRCA1 polypeptides with amino acid substitutions at a position homologous to S988 of human BRCA1, for example alanine or glutamic acid substitutions. The application further discloses methods for determining exposure to genotoxic stress, by determining whether a residue homologous to S988 of human BRCA1 in a cell is phosphorylated. In particular examples, antibodies immunologically specific for BRCA1 polypeptides containing a phosphorylated S988 may be used to assist in determining whether S988 is phosphorylated.

### EXAMPLE 1

### hCds1 Phosphorylates a BRCA1-Derived Peptide

To demonstrate that human hCds1 directly phosphorylates BRCA1, six glutathione S-transferase (GST)-BRCA1 fusion proteins containing overlapping BRCA1 fragments were used as substrates for recombinant hCds1 in in vitro kinase assays (Table 1).

TABLE 1
hCds1 directly phosphorylates BRCA1.

| BRCA1 fragment (amino acids) | Phosphorylation by hCds1 |
|---|---|
| GST-BRCA1.1 (1-324) | − |
| GST-BRCA1.2 (260-553) | − |
| GST-BRCA1.3 (502-802) | − |
| GST-BRCA1.4 (758-1064) | +++ |
| GST-BRCA1.5 (1005-1313) | − |
| GST-BRCA1.6 (1314-1863) | − |

GST-BRCA1 fusion proteins were prepared as described by Scully et al. (Cell 88:265-75, 1997). GST-BRCA1.1 represents GST fused to amino acid residue numbers 1-324 of BRCA1 (residue number 1 being the N-terminal residue of BRCA1). GST-BRCA1.2 through GST-BRCA1.5 represents GST fused to BRCA1-derived polypeptides progressively closer to the BRCA1 C-terminus. GST-BRCA1.6 includes the BRCA1 residues closest to the C-terminus (residues 1314-1863).

The in vitro kinase assays were performed in a 15 μl total volume with 1 μg of recombinant hCds1 (prepared and purified as described in Brown et al., PNAS 96:3745-50, 1999; hereinafter Brown et al.) and 2 μg of the GST-BRCA1 fusion protein indicated in the left column of Table 1. The reaction buffer contained 10 mM HEPES pH 7.5, 75 mM KCl, 5 mM MgCl₂, 0.5 mM EDTA, 1 mM dithiothreitol, 100 μM ATP and 5 μCi of [γ-³²P]ATP. The mixture was incubated at 30° C. for 15 minutes then electrophoresed in a denaturing polyacrylamide gel as described in Lee et al. (Nature 404:201-4, 2000, herein, Lee et al.). Extent of phosphorylation was determined semiquantitatively by autoradiography.

Of the six fusion proteins, hCds1 phosphorylated most strongly GST-BRCA1.4 (representing GST fused to BRCA1 amino acids 758-1,064, approximately in the center of the BRCA1 protein). None of the other five GST-BRCA1 fusion proteins were significantly phosphorylated by hCds1.

### EXAMPLE 2

### Endogenous hCds1 From Irradiated Breast Cancer Cells Phosphorylates BRCA1

To demonstrate that hCds1 phosphorylates BRCA1 in cells, endogenous hCds1 was obtained from the breast cancer-derived cell line MCF-7 before and after treatment of MCF-7 cells with 10 Gy of gamma irradiation. This amount of irradiation activates BRCA1-dependent DNA damage repair pathways. The pre- and post-irradiation hCds I preparations were then tested for their ability to phosphorylate GST-BRCA1.4.

Endogenous hCds1 was obtained by immunoprecipitation with an affinity-purified antibody specific for hCds1 (the preparation and characteristics of the antibody are described in Brown et al.). To determine the effect of gamma irradiation on hCds1 activity, hCds1 was immunoprecipitated with 1 μg of preimmune serum or 1 μg of from MCF-7 cell lysate (10⁷ cells) prepared either before or 1 hour after gamma irradiation (10 Gy). The kinase reactions were performed as described in Example 1, using GST-BRCA1.4 as kinase substrate.

Preimmune serum was used to immunoprecipitate lysates from four separate sets of MCF-7 cells, two of which had been gamma irradiated, and two of which had not been irradiated. The immunoprecipitates were used as a potential source of hCds1 in a kinase assay. If hCds1 kinase activity is present in the immunoprecipitate, then GST-BRCA1.4 should become phosphorylated. None of the immunoprecipitates contained hCds1 kinase activity, because they could not incorporate phosphate into GST-BRCA1.4. This is the appropriate control result: preimmune serum lacks the ability to immunoprecipitate hCds1 from cell lysates, regardless of whether the lysates contained active hCds1 or not.

The same experiment was repeated, except that instead of preimmune serum, an anti-hCds1 antibody is used to perform the immunoprecipitation. A strong band of phosphorylation was observed when gamma irradiated cells were immunoprecipitated using anti-hCds1. However, only a minimal band was observed when unirradiated cells are immunoprecipitated, or when GST-BRCA1.4 was omitted from the kinase reaction. The immunoblot for hCds1 showed a strong band, demonstrating that the immunoprecipitation procedure was successful in isolating the hCds1 protein.

These results demonstrate that irradiation of MCF-7 cells induces a kinase that phosphorylates GST BRCA1.4 which can be precipitated with an anti-hCds1 antibody. The ability of hCds1 inmmunoprecipitated from irradiated cells to phosphorylate GST-BRCA1.4 was almost 6-fold higher than hCds1 immunoprecipitated from unirradiated cells. Thus, irradiation activates endogenous hCds1 kinase activity in breast cancer cells, resulting in phosphorylation of the same BRCA1-derived peptide that was phosphorylated by recombinant hCds1.

### EXAMPLE 3

### HCds1 Phosphorylates BRCA1 at Serine 988

To identify which amino acid(s) of BRCA1.4 was phosphorylated, mass spectrometric analysis was performed on hCds1-phosphorylated GST-BRCA1.4. It was determined that Ser 988 of BRCA1 was the phosphorylated amino acid. Ser 988 of human BRCA1 aligns with Ser 971 of mouse and Ser 987 of dog BRCA1. Table 2 shows the homologous regions of mouse, dog and human BRCA1, aligned to show homology. Each of the three species has a serine residue at the position corresponding to Ser 988 of human BRCA1. It is likely that the corresponding residues in the mouse and dog or other species are targets for Cds1 phosphorylation of BRCA1.

TABLE 2
Alignment of Partial BRCA1 sequences

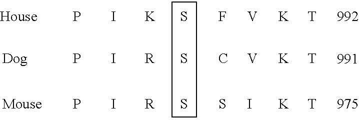

An in vitro kinase reaction was performed using as substrates GST-BRCA1.4 and GST-BRCA1.4 (S988A) in which Ser 988 was changed to alanine. The reaction was performed as described in Examples 1 and 2. Recombinant hCds1 (expressed and purified as a GST fusion protein) was used as a source of kinase activity. The ability of recombinant hCds1 to phosphorylate GST BRCA1.4 and GST-BRCA1.4 (S988A) was compared. Because of this mutation, the BRCA1.4 fragment can not be phosphorylated by hCds1.

In the presence of GST BRCA1.4 or GST BRCA1.4 (S988A) alone (i.e. hCds1 is absent), no phosphorylation was observed. This result was expected because no kinase was present.

When both hCds1 and GST-BRCA1.4 were present, a band was present in both ³²P lanes, indicating that both hCds1 and GST BRCA1.4 were phosphorylated. This result establishes that hCds1 phosphorylates GST BRCA1.4, confirming data presented in Examples 1 and 2.

When both hCds1 and GST-BRCA1.4 (S988A) are present, hCds1 showed evidence of autophosphorylation, indicating that the enzyme is active. However, there was no phosphorylation of the substrate GST-BRCA1.4 (S988A). Thus, even though hCds1 was active, it could not phosphorylate the mutant BRCA1 peptide.

Thererfore, activated hCds1 phosphorylates BRCA1 at serine 988, and cannot phosphorylate BRCA1 when S988 is mutated to alanine.

### EXAMPLE 4

### BRCA1 Is Phosphorylated at S988 in Vivo

To demonstrate that BRCA1 S988 is phosphorylated in vivo, a polyclonal antibody (anti-S988-P) was generated that was specific for phosphorylated Ser 988. S988-P (amino acid sequence CRIPPLFPIKSFVKTK, SEQ ID NO. 1) was synthesized, in which the Ser residue (corresponding to S988) was phosphorylated. It was conjugated by its NH₂-terminal cysteine to keyhole limpet hemocyanin (KLH; Fluka) using the heterobifunctional cross-linker, n-maleimidobenzoyl-N-hydroxysuccinimde ester (sulpho-MBS, Pierce), and referred to herein as S988-P KLH conjugate.

New Zealand white rabbits were immunized with 1 mg S988-P KLH conjugate suspended in complete adjuvant, and boosted with 0.3 mg S988-P KLH conjugate in incomplete adjuvant at four and seven weeks following the first immunization. Anti-S988-P antibody was purified using an affinity column to which S988-P had been attached using activated CH-Sepharose 4B according to the manufacturer's instructions (Pharmacia). The specificity of the anti-S988-P antibody was demonstrated by showing that it does not recognize the GST-BRCA1 (S988A) mutant. Using the anti-S988-P antibody, the phosphorylation status of BRCA1 was examined in AT⁺ and AT⁻ cells that were gamma irradiated.

Irradiation-induced activation of hCds1 proceeds through ATM (ataxia telangiecstasia mutated), a phosphoinositide-activated kinase. Cell lines possessing functional ATM are referred to as AT⁺, whereas those lacking functional ATM are referred to as AT⁻. AT⁺ cells respond to gamma irradiation and other forms of genotoxic stress by phosphorylating hCds1, thereby activating hCds1-dependent signaling pathways. However, in AT⁻ cells, gamma irradiation does not activate hCds1; consequently, irradiation fails to activate hCds1-dependent signaling pathways. Similarly, the human embryonic kidney cell line 293T is stably transformed to express functional ATM, and has an AT⁺ phenotype.

AT⁺, AT⁻, and 293T cells were treated with irradiation (2.5 Gy), or mock-irradiated. Immunoblots (Western blots) were prepared from total cell lysates using standard techniques (for example, as described in Ausubel et al., Short Protocols in Molecular Biology, Fourth Edition 1999). The blots were probed with the anti-S988-P antibody described above, and with anti-BRCA1 antibodies (monoclonal Ab-3, Calbiochem, or rabbit polyclonal, BRCA1 1847-1863, Pharmingen).

No phosphorylation of S988 was observed in AT⁻ cells, regardless of whether the cells were irradiated. The lack of phosphorylation was not due to lack of BRCA1 in the cells, since immunoreactive BRCA1 was observed. In contrast, irradiation induced S988 phosphorylation in AT⁺ and 293T cells. Thus, irradiation results in ATM-dependent BRCA1 S988 phosphorylation in vivo.

Notably, a low level of S988 phosphorylation was observed in unirradiated AT⁺ cells and 293T cells, but not in unirradiated AT⁻ cells. This result indicates that the ATM-hCds1-BRCA1 signaling pathways is partially active in the basal (unirradiated) state. In addition, the S988-P antibody detected a single band whereas the BRCA1 antibody detected a more diffuse band. This indicates that irradiation-induced DNA damage may cause phosphorylation of BRCA1 sites other than S988.

### EXAMPLE 5

### Radiation-Induced BRCA1 Phosphorylation at S988 is hCds1-Dependent

To confirm that radiation-induced S988 phosphorylation occurred through hCds1-dependent pathways, S988 phosphorylation was investigated in cells expressing a kinase-dead hCds1 mutant (KD). The KD mutant is a hCds1-derived polypeptide that is mutated in its active site (lysine 249 mutated to arginine; Brown et al., PNAS 96:3745-50, 1999), and is therefore incapable of phosphorylating hCds1 substrates. However, the mutation does not impair hCds1 substrate binding. Thus, KD acts as a dominant negative, that is, an in vivo competitive inhibitor of hCds1 kinase activity.

To demonstrate that expression of KD would block phosphorylation of S988 in BRCA1, 293T cells were mock-transfected; transfected with an empty vector, or transfected with a vector expressing KD. The cells were irradiated (or not), and cell lysates prepared and assayed as described in Example 4.

Irradiation induced phosphorylation of S988 in mock-transfected cells, and in cells transfected with empty vector. However, KD expression blocked radiation-induced S988 phosphorylation. In unirradiated cells transfected with empty vector, a low level of background S988-P was detected. These experiments demonstrate that radiation-induced BRCA1 phosphorylation at S988 is hCds1-dependent.

### EXAMPLE 6

### BRCA1 and hCds1 Colocalize In Nuclear Foci

BRCA1 forms nuclear foci that disperse after DNA damage (Scully et al., Cell 90:425-35, 1997; Thomas et al., Cell Growth Differ. 8:801-9, 1997). An immunofluorescence study was performed to determine whether hCds1 exhibited similar subcellular localization, and whether phosphorylation at S988 affected BRCA1's ability to disperse from nuclear foci in response to gamma irradiation.

Immunofluorescence staining of BRCA1 and hCds1 was performed as described in Zhong et al., Science 285:747-50, 1999; Wilson et al., Nature Genetics 21:236-40, 1999; and Lee et al., Nature 404:201-4, 2000. Confocal laser scanning microscopy was performed with a Noran Odyssey real-time laser confocal microscope equipped with a Nikon Diaphot inverted microscope.

In unirradiated cells, immunofluorescence staining of MCF-7 cells demonstrated that hCds1, like BRCA1, exists in nuclear foci. Double staining of BRCA1 and hCds1 demonstrated that BRCA1 foci and hCds1 foci colocalize in the nucleus.

One hour after gamma irradiation (20 Gy), the BRCA1 nuclear foci disperse, whereas the hCds1 nuclear foci showed less evidence of dispersion. Consequently, the two foci do not colocalize, indicating that gamma irradiation triggers the release of BRCA1 from hCds1 foci.

To determine whether S988 phosphorylation was involved in BRCA1 dispersion, MCF-7 cells were transfected with DNA sequences expressing a BRCA1-hemagglutinin fusion protein (HA-BRCA1), or a BRCA1-hemagglutinin fusion protein in which serine 988 was mutated to alanine (HA-BRCA1[S988A]). Immunofluorescence with hCds1 antibody and anti-HA antibody was performed on irradiated and unirradiated cells.

In the absence of gamma irradiation, both HA-BRCA1 and HA-BRCA1 [S988A] colocalized with endogenous hCds1. However, in response to gamma irradiation, HA-BRCA1 dispersed strongly whereas HA-BRCA1 [S988A] dispersed poorly and remained colocalized with hCds1, indicating that phosphorylation of Ser 988 is important for BRCA1 dispersion after gamma irradiation.

### EXAMPLE 7

### Interaction of hCds1 and BRCA1

BRCA1 and hCds1 form a Complex in Unirradiated Cells that Dissociates Upon Exposure to Ionizing Radiation

To further demonstrate that Cds1 and BRCA1 interact, and that gamma irradiation affects this interaction, co-immunoprecipitation studies were performed.

Plasmids expressing BRCA1 fused to the epitope tag myc (myc-BRCA1) and hCds1 fused to the epitope tag V5 (V5-hCds1 WT) were constructed using standard molecular biology techniques. In addition, a plasmid expressing the kinase-dead hCds1 mutant fused to V5 (V5-hCds1 KD) was constructed (Lee et al.). Plasmids were cotransfected into MCF-7 cells in various combinations, and the impact of irradiation on the hCds1-BRCA1 interaction was determined through co-immunoprecipitation.

Cells cotransfected with myc-BRCA1 and V5-hCds1 WT, but were unirradiated, were lysed, and anti-myc and anti-V5 antibodies used to immunoprecipitate the lysates. After immunoprecipitation, standard immunoblots were performed using anti-V5 and anti-myc antibodies for detection. When anti-myc or anti-V5 antibodies were used for immunoprecipitation, both proteins were immunoprecipitated. Thus, in unirradiated cells, BRCA1 and hCds1 form a complex.

Cells cotransfected with BRCA1 and hCds1 WT were irradiated (10 Gy) prior to immunoprecipitation. In these irradiated cells, the anti-myc antibody immunoprecipitated myc-BRCA1, but not V5-hCds1. Similarly, the anti-V5 antibody immunoprecipitates V5-hCds1, but not myc-BRCA1. This shows that in irradiated cells, BRCA1 and hCds1 do not form a complex that can be detected by co-immunoprecipitation.

When V5-hCds1 was omitted as a control, immunoprecipitation with anti-myc antibody immunoprecipitated only myc-BRCA1. Immunoprecipitation with anti-V5 did not immunoprecipitate either protein.

Cells cotransfected with myc-BRCA1 and V5-hCds1 KD, but were not irradiated, were lysed and immunoprecipitated. Myc-BRCA1 and V5-hCds1 KD co-immunoprecipitated with both anti-myc and anti-V5 antibodies. This indicates that myc-BRCA1 and V5-hCds1 KD also form a complex in unirradiated cells. In addition, after irradiation, myc-BRCA1 and V5-hCds1 KD continued to co-immunoprecipitate. Thus, unlike the wild type hCds1, the kinase-dead hCds1 did not induce BRCA1 to disassociate from it after irradiation. Therefore, an hCds1-dependent event may be required for radiation-induced dissociation of hCds1 and BRCA1.

Endogenous BRCA1 and hCds1 reacted in the same manner as the epitope-tagged proteins. Lysates from unirradiated and irradiated MCF-7 cells were immunoprecipitated with anti-hCds1 and anti-BRCA1 antibodies. In unirradiated cells, the two proteins co-immunoprecipitated, whereas in irradiated cells, no complex formation was observed. This confirms that endogenous BRCA1 and hCds1 interact in MCF-7 cells and separate after gamma irradiation.

Phosphorylation of BRCA1 is Involved in the Release from hCds1

Because the kinase activity of hCds1 was shown to be involved in the release of BRCA1, it was next demonstrated that phosphorylation of BRCA1 by hCds1 was involved in triggering BRCA1 release. Co-immunoprecipitation experiments with hCds1 and BRCA1 [S988A] were performed to show that the nonphosphorylatable BRCA1 mutant could release from hCds1.

A plasmid expressing BRCA1 fused to a hemagglutinin (HA) epitope tag was constructed using standard molecular biology methods. Similar plasmids were constructed expressing two BRCA1 S988 mutants: HA-BRCA1 [S988A], containing the nonphosphorylatable alanine mutation at 988; and HA-BRCA1 [S988E], which substitutes a glutamic acid for serine at position 988. The negatively-charged glutamic acid substitution functions in a similar manner to phosphorylated serine, and therefore HA-BRCA1 [S988E] may be regarded as a BRCA1 mutation that results in a “perpetually phosphorylated” residue at position 988.

Plasmids expressing HA-BRCA1 WT, HA-BRCA1 [S988A], or HA-BRCA1 [S988E] were cotransfected with a plasmid expressing V5-hCds1. After transfection, MCF-7 cells (unirradiated or irradiated with 10 Gy) were lysed and immunoprecipitated with anti-HA and anti-V5 antibodies. Western blots were prepared, and the presence of the V5-hCds1 and HA-BRCA1 proteins in immunoprecipitates was assessed. An anti-HA antibody was used to prepare the immunoprecipitates.

When wild type HA-BRCA1 was coexpressed with V5-hCds1, and the cells not irradiated, the two proteins co-immunoprecipitated; immunoprecipitation with the anti-HA antibody resulted in a strong band in both the HA and V5 immunoblots. However, upon irradiation the band in the V5 immunoblot lane was lost, showing that V5-hCds1 was no longer found in a co-immunoprecipitable complex with HA-BRCA1.

When the nonphosphorylatable HA-BRCA1 [S988A] was coexpressed with V5-hCds1, and the cells not irradiated, the two proteins co-immunoprecipitated. However, upon irradiation HA-BRCA1 [S988A] failed to disassociate from V5-hCds1. The band in the V5 immunoblot lane remained strong, showing that V5-hCds1 remained complexed to HA-BRCA1 [S988A]. Thus, the presence of the S988A mutation prevents radiation-induced dissociation of BRCA1 from hCds1.

When HA-BRCA1 [S988E] was coexpressed with V5 hCds1, and the cells not irradiated, the two proteins co-immunoprecipitated. This result indicated that a negative charge at BRCA1 position 988 (the type conferred by a glutamic acid) was not in itself sufficient to disassociate BRCA1 from hCds1. In irradiated cells, V5-hCds1 was no longer observed in a co-immunoprecipitable complex with HA-BRCA1 [S988E]. Hence a negative charge at BRCA1 988 (such as that conferred by gum or a phosphorylated serine) is necessary but not sufficient for dissociation of the hCds1-BRCA1 complex.

Modification of hCds1 is Involved in Radiation-Induced hCds1-BRCA1 Dissociation

After irradiation and other forms of DNA damage, hCds1 was autophosphorylated. To demonstrate that hCds1 autophosphorylation was involved in the radiation-induced dissociation of BRCA1 from hCds1, the experiments described above were repeated with the kinase dead hCds1 mutant substituting for wild type (V5-hCds1 [KD]).

When nonphosphorylatable HA-BRCA1 [S988A] was coexpressed with V5-hCds1 [KD], the proteins remained in a co-immunoprecipitable complex in both unirradiated and irradiated cells. Similar results were observed when HA-BRCA1 [S988E] was coexpressed with V5 hCds1 KD. This latter result contrasts sharply with the result when the wild type, kinase-active hCds1 is present; irradiation induces HA-BRCA1 [S988E] to disassociate from hCds1. Thus, modification of both hCds1 and BRCA1 is involved in their separation.

### EXAMPLE 8

### S988 Phosphorylation Promotes DNA Damage Repair by BRCA1

HCC1937 breast cancer cells, which carry a homozygous mutation in BRCA1, are extremely sensitive to DNA damage. Exogenous expression of BRCA1 restores resistance to DNA damage in these cells (Zhong et al., Science 285:747-50, 1999). To demonstrate that hCds1 activity and S988 phosphorylation are involved in this improved resistance to DNA damage, the following methods were performed.

HCC1937 cells (BRCA negative) were transfected with an empty vector as described in Zhong et al., or with vectors expressing HA-BRCA1 wild type, [S988A], or [S988E], as described in Lee et al. Forty-eight hours after transfection, cells were either gamma irradiated with 0.14 Gy, or left unirradiated. Eight days after irradiation, cell survival was determined.

Only about 3% of cells transfected with empty vector survived 8 days after irradiation. Cells transfected with HA-BRCA1 [S988A] showed no significant improvement in survival. However, survival improved about 3-fold in cells transfected with wild type BRCA1 or the S988E mutant.

This result shows that hCds1 phosphorylation of S988 is involved in promoting BRCA1's role in DNA damage repair. A mutation at S988 renders the BRCA1 protein nonphosphorylatable by hCds1 and reduces its ability to repair the cell after exposure to genotoxic agents. Consequently, tumor cell survival after exposure to such agents is significantly impaired.

### EXAMPLE 9

### S988 Phosphorylation Regulates BRCA1 Transcriptional Activity

To determine whether BRCA1's ability to promote p21waf1/cip1 expression was influenced by S988 phosphorylation, the activity of the p21 promoter was studied in irradiated and unirradiated cells transfected with wild type BRCA1, BRCA1 S988A or BRCA1 S988E. The S988A mutation renders BRCA1 nonphosphorylatable at this position, whereas the S988E mutation irreversibly confers a negative charge at position 988, thereby mimicking the effect of phosphorylation.

CV1 cells were cotransfected with plasmids expressing BRCA1, BRCA1 mutants, or empty vector control, together with a plasmid containing a luciferase cDNA operably linked to the promoter region of p21waf1/cip1. Transfections were carried out using the Effectene transfection kit (Qiagen), with 0.02 μg of the p21-luciferase reporter plasmid, and 0.18 μg of BRCA1 or BRCA1 mutant plasmid, or empty vector control (pcDNA3.1, Invitrogen). An activated p21 promoter expresses functional luciferase, which may be quantified in a luciferase assay system (Promega) 48 hours after transfection. Thus the ability of BRCA1 or BRCA1 mutants to activate the p21 promoter is quantitatively reflected by the luciferase activity present in the cell lysate.

Lysates of cells transfected with empty vector, either unirradiated or irradiated, showed no significant luciferase activity, indicating that the p21 promoter is not activated in the absence of exogenous BRCA1.

Luciferase activity was observed in unirradiated cells transfected with wild-type BRCA1, about 30-fold higher than basal activity observed in cells transfected with empty vector. Irradiation increased the luciferase activity about another 10-fold, to 400 times basal activity.

Luciferase activity in unirradiated cell lysates from cells transfected with BRCA1 S988A was increased about 30-fold over basal activity, and was at the same level observed in unirradiated cells transfected with wild type BRCA1. However, after irradiation, luciferase activity in S988A-transfected cells increased only modestly. Thus, the S988A mutation significantly impairs the ability of BRCA1 to activate the p21 promoter in response to irradiation.

Luciferase activity in lysates from cells transfected with BRCA1 S988E was 500-fold higher than basal activity in unirradiated cells, and was approximately equivalent to that observed in irradiated, wild type BRCA1-transfected cells. Irradiation did not further increase luciferase activity. Thus, the S988E mutation markedly activates BRCA1-stimulated transcription from the p21 promoter. Quantitatively, the observed effect is similar to the activating effect of radiation in cells transfected with wild type BRCA1.

### EXAMPLE 10

### S988 Phosphorylation Regulates BRCA1 Interaction with Transcription Repressors

CtIP, as part of any a corepressor complex with CtBP, binds to BRCA1 and suppresses its ability to activate transcription. In response to ionizing radiation, CtIP dissociates from BRCA1, allowing BRCA1 to activate transcription. The molecular mechanism of this radiation-induced dissociation was previously unknown. The experiments disclosed herein demonstrate that radiation-induced phosphorylation of BRCA1 S988 promotes this dissociation.

293T cells (0.5×10⁷) were transfected with 10 μg of plasmid expressing HA-BRCA1, HA-BRCA1 S988A, HA-BRCA1 S988E, or empty vector (pcDNA3.1, Invitrogen). These were cotransfected with CtIP and CtBP expression vectors (7.5 μg and 2.5 μg, respectively), using SuperFect Transfection Reagent (Qiagen) in the presence of 10 nM estradiol. After 36-48 hours, cells were Irradiated (15 Gy) or left unirradiated. Cell lysates were prepared 1-2 hours later.

CtIP was immunoprecipitated from the cell lysates with 0.5 μg of anti-CtIP antibody. Using a standard immunoblotting protocol (see Ausubel et al., In Current Protocols in Molecular Biology, Greene Publ. Assoc. and Wiley-Intersciences, 1992), the immunoprecipitates were resolved by SDS-PAGE and transferred to a nitrocellulose membrane. The presence of BRCA1 was determined by immunoblotting with mouse anti-HA antibody, which binds to the HA epitope tag of the BRCA1 fusion proteins used herein (Babco Inc.). The presence of CtIP in the immunoblot was confirmed by immunoblotting with anti-CtIP antibody.

In unirradiated cells transfected with wild-type HA-BRCA1, immunoprecipitation with anti-CtIP antibody co-precipitated HA-BRCA1, but in irradiated cells, little or no HA-BRCA1 co-precipitated. Thus, CtIP formed a complex with BRCA1, and irradiation induced CtIP to dissociate from BRCA1.

In contrast to cells transfected with wild type HA-BRCA1, the S988A mutant remained complexed to CtIP even in irradiated cells. With the mutant HA-BRCA1 S988E, little complex formation was observed with CtIP in both irradiated and unirradiated cells.

Taken together, these results demonstrate that irradiation-induced S988 phosphorylation plays a significant role in regulating the CtIP-BRCA1 interaction. Specifically, S988 phosphorylation promotes dissociation of CtIP from BRCA1, thereby enhancing BRCA1 transcriptional activity.

### EXAMPLE 11

### S988 Phosphorylation Mediates

### BRCA1 Regulation of Estrogen-Dependent Transcription

In estrogen responsive cells, the transcription of several genes is regulated in part through regulatory DNA sequences termed estrogen-responsive promoters. Estrogen responsiveness of estrogen-responsive promoters is largely conferred through estrogen response element (ERE), which are DNA sequences that bind the estrogen receptor-estradiol complex. Transcription from the ERE in estrogen-responsive promoters is suppressed by BRCA1 (Fan et al., Science: 284:1354-6, 1999). This is in contrast to the p21waf1/cip1 promoter, where BRCA1 activates transcription (see Example 10). Using the methods disclosed herein, the physiologic significance and molecular mechanism of ERE suppression by BRCA1 is revealed.

To determine whether S988 phosphorylation mediates BRCA1 regulation of ERE, SaOS2 or U20S cells were transfected with a plasmid containing a luciferase cDNA operably linked to an ERE (0.08 μg). To establish an estrogen-responsive phenotype, the cells were also transfected with a plasmid expressing a functional estrogen receptor (ERa; 0.08 μg). In addition, cells were cotransfected with plasmids expressing wild type HA-BRCA1, HA-BRCA1 [S988A], HA-BRCA1 [S988E], or empty vector. Transfection was performed using the Effectene transfection kit (Qiagen). After transfection, cells were incubated for 16-24 hours, then 10 nM estradiol or control solution was added, and the cells were incubated for another 24 hours. Cells were then lysed, and luciferase activity determined using the dual luciferase assay (Promega). Luciferase activity was normalized for transfection efficiency in each case.

In cells transfected with ERa and empty vector (and thus do not express a BRCA1 construct), basal luciferase activity was low, but increased over 10-fold upon addition of E2. Thus, the assay system accurately responds to E2 by increasing transcription from ERE-containing promoters.

In E2-treated cells transfected with ERa and HA-BRCA1, luciferase activity was only modestly elevated from basal levels. Compared to cells transfected with empty vector, luciferase activity was reduced by about 5-fold. Similarly, E2-treated cells transfected with ERa and HA-BRCA1 [S988E] showed low luciferase activity in spite of E2 treatment. Thus, expression of HA-BRCA1 or S988E reduced transcription from ERE-containing promoters.

In E2-treated cells transfected with ERa and HA-BRCA1 [S988A], luciferase activity remained high in spite of E2 treatment. This result contrasts with the result observed for cells transfected with wild type HA-BRCA1. Thus, the S988A mutant failed to reduce transcription from ERE-containing promoters.

Similar results were observed using U2OS cells.

If S988 participates in the BRCA1-dependent suppression of ERa-dependent transcription pathways, then increasing S988 phosphorylation of endogenous BRCA1 by overexpressing hCds1 should suppress ERa activity. To examine the effect of hCds1, 0.1 μg of ERE-luciferase reporter, 0.1 μg of hCds1 expression plasmid (expressing either the wild-type or kinase-dead mutant), or empty vector (pcDNA 3.1) and 0.1 μg of ERa expression vector were transfected into the SaOS2 cells. After transfection, cells were treated with 10 nM estradiol (E2) or control solution, and harvested 48 hours later. Luciferase activity was determined as described above, and luciferase activity with E2 was divided by luciferase activity without E2 to calculate E2-inducibility.

Cells transfected with either empty vector or the kinase-dead hCds1 mutant showed about 12-fold inducibility by E2. However, cells transfected with wild type hCds1, showed only about 2- to 3-fold inducibility by E2. Thus, the kinase activity of hCds1 significantly enhanced the ability of BRCA1 to “shut off” or inhibit transcription mediated by the E2-ERa complex. Since hCds1 interacts with BRCA1 primarily by phosphorylating S988 (Lee et al.) this result demonstrates that S988 phosphorylation mediates BRCA1 regulation of estrogen-stimulated transcription. More specifically, S988 phosphorylation by hCds1 reduces the ability of BRCA1 to inhibit estrogen-stimulated transcription.

### EXAMPLE 12

### S988 Phosphorylation Inhibits Complex Formation Between BRCA1 and ERa

BRCA1 may suppress ERa activity by inhibiting its function through direct or indirect interaction. To determine if there was a direct interaction between BRCA1 and ERa, the following methods were utilized.

293T cells were transfected with a plasmid expressing either HA-BRCA1 or an empty vector. Cell lysates were immunoprecipitated with an anti-HA antibody (rabbit, Babco). The immunoprecipitates were resolved by SDS-PAGE and transferred to a nitrocellulose membrane. After allowing protein renaturation, the membrane was blocked with 5% milk in PBS for 30 minutes and then incubated overnight at 4° C. in binding buffer (0.05% NP-40, 1% skim milk in PBS) containing 20 μg/ml ERa (Affinity Bioreagents; seeYohannon et al., J. Biol. Chem. 274:18769-76, 1999 for additional description of this method). Bound ERa was detected by immunoblotting with mouse anti-ERa antibody (Neomarkers).

In cells transfected with HA-BRCA1, immunoprecipitation with anti-HA antibody yielded a protein of the same size as HA-BRCA1. That protein bound ERa, because it was detected not only with anti-HA antibody, but with an anti-ERa antibody. In cells transfected with empty vector, no detectable protein was immunoprecipitated. This result demonstrates that BRCA1 can directly interact with ERa.

BRCA1-ERa interaction in vivo was examined by coimmunoprecipitation before and after exposure to ionizing radiation. 293T cells (1×10⁷) were cotransfected with plasmids expressing ERa and empty vector or HA-BRCA1 (wild type, S988A mutant, or S988E mutant). After transfection (48 hours), cells were irradiated with 15 Gy or left unirradiated, and 1-2 hours later, cell lysates were prepared for immunoprecipitation. Immunoprecipitation was performed with anti-ERa antibody, and immunoblotting was performed with anti-HA antibody. Thus, if HA-BRCA1 formed an in vivo complex with ERa, it would be immunoprecipitated with the anti-ERa antibody, and a band would be present on the anti-HA immunoblot.

Cells were transfected with ERa and empty vector and immunoprecipitated with anti-ERa antibody. The ERa immunoblot revealed a band, indicating that the immunoprecipitation protocol was functional. However, the anti-HA immunoblots showed no band, indicating that no HA-containing protein was present. Irradiation had no effect on the result.

Cells were transfected with ERa and HA-BRCA1 [S988A] and immunoprecipitated with anti-ERa antibody. The anti-HA immunoblots revealed a band in unirradiated cells that intensified after irradiation. However, the bands were more intensified when cells were transfected with wild type HA-BRCA1, and irradiation produced a much more striking increase. When cells were transfected with the S988E mutant, anti-HA immunoblots revealed intense bands in both unirradiated and irradiated cells.

Therefore, BRCA1 and ERa interact directly in vitro and in vivo, and this interaction is enhanced by DNA damaging agents such as ionizing radiation. Moreover, the results demonstrate that S988 phosphorylation mediates the interaction. Specifically, when S988 is phosphorylated by hCds1 after exposure to DNA damaging agents, BRCA1 binding to ERa is enhanced. In this manner, DNA damaging agents induce BRCA1 to inhibit estrogen-stimulated transcription.

### EXAMPLE 13

### BRCA1 Inhibits Interaction of ERa With Nuclear Receptor Coactivators

By interacting with ERa, BRCA1 may affect ERa's interaction with other proteins. Nuclear receptors activate transcription by recruiting one of many coactivators, including histone acetyltransferases such as PCAF. ERa interacts with PCAF, and its interaction with PCAF promotes its transcriptional activity (Blanco et al., Gene. Devel. 12:1638-51, 1998). To the extent that BRCA1 binds ERa, it may prevent ERa's interaction with PCAF and like coactivators, thereby reducing ERa-mediated transcription. This may be particularly true after damage-induced S988 phosphorylation of BRCA1.

To evaluate the effect of IR on ERa-PCAF interaction, coimmunoprecipitation experiments were performed. COS-7 cells were cotransfected with plasmids expressing ERa, PCAF linked to the epitope tag FLAG (PCAF-FLAG), and HA-BRCA1 (wild type, S988A, or S988E mutant). Following transfection (48 hours), cells were irradiated with 15 Gy or left unirradiated, and 1-2 hours later, cell lysates were prepared for immunoprecipitation. Cell lysates were immunoprecipitated with anti-ERa or anti-FLAG antibody, and immunoblots were probed with anti-ERa or anti-FLAG antibody.

In cells transfected with ERa, FLAG-PCAF, and wild type HA-BRCA1 which were not irradiated cells, a strong band was observed after immunoprecipitation with anti-ERa and immunoblotting with anti-FLAG. This demonstrates that PCAF and ERa form a complex. However, the band was considerably weaker after radiation, showing that radiation treatment inhibited this interaction.

In cells transfected with ERa, FLAG-PCAF, and HA-BRCA1 [S988A], the S988A mutant continued to form a complex with PCAF before and after irradiation. In contrast, the S988E mutant formed a relatively weak complex even in the absence of radiation treatment. Therefore, DNA damage reduces ERa interaction with nuclear coactivators, an effect that is BRCA1-mediated and influenced by the phosphorylation status of S988.

### EXAMPLE 14

### Phosphorylation of BRCA1 S988 Mediates Interaction of Estrogen Receptors With Estrogen-Response Elements

ERa affects transcription of estrogen-responsive promoters by binding to the ERE. By using electrophoretic mobility shift assay, ERa binding to oligonucleotides including the ERE was evaluated. Nuclear extracts were prepared from irradiated (20 Gy) and unirradiated SaOS2 cells expressing ERa and HA-BRCA1 (wild type, S988A, or S988E mutant). The ability of these extracts to retard the electrophoretic mobility of labeled ERE oligonucleotide probes was determined.

ERE probes were prepared by filling in the end of annealed oligonucleotide (5′- AGCTTGGTCACTGTGACCG-3′, SEQ ID NO: 2 and 5′-CATCCGGTCACAGTGACCA-3′ SEQ ID NO: 3) with Klenow enzyme, [α-³²P]-ATP, and unlabeled dNTPs. The nuclear extracts were bound to radiolabeled probes at room temperatures, and run on a polyacrylamide gel using standard mobility-shift protocols such as those described in Ausubel et al. (In Current Protocols in Molecular Biology, Greene Publ. Assoc. and Wiley-Intersciences, 1992).

Nuclear extracts from cells transfected with empty vector were used to bind labeled ERE. When ERa was expressed, the mobility of ERE was retarded compared to unbound ERE when no ERa was expressed. Thus, the labeled ERE formed a nucleoprotein complex with ERa, thereby reducing the ERE's electrophoretic mobility.

When HA-BRCA1 wild type was expressed, the ability of ERa to bind to ERE and retard its mobility was diminished significantly after treatment with ionizing radiation (IR). In the presence of BRCA1 [S988A], the ability of ERa to bind to the ERE was not affected by IR. In the presence of BRCA1 [S988E], ERa bound poorly to the ERE even in the absence of IR.

### EXAMPLE 15

### Production of Sequence Variants

Disclosed herein methods for modifying sensitivity to genotoxic stress by administration of a biological macromolecule, such as a protein, that alters BRCA1 phorphorylation by Cds1. It is understood by those skilled in the art that use of non-native protein sequences (such as polymorphisms, fragments, or variants) can be used to practice the methods of the present disclosure, as long as the distinctive functional characteristics of the protein are retained.

For example, Cds1 variants can be used to practice the methods disclosed herein if they retain their ability to alters BRCA1 phorphorylation by Cds1. This activity can readily be determined using the assays disclosed herein. For example, BRCA1 variants and fragments can be tested for its reduced ability to disperse from nuclear foci according the methods described in Example 6, impaired enhancement of p21-mediated expression following irradiation according to Example 9, and/or persistent complex formation with CtIP following irradiation according to Example 10. BRCA1 variants that exhibit one or more of these characteristics will, upon delivery to tumor cells, enhance their sensitivity to genotoxic agents.

This disclosure facilitates the use of DNA molecules, and thereby proteins, derived from a native protein but which vary in their precise nucleotide or amino acid sequence from the native sequence. Such variants can be obtained through standard molecular biology laboratory techniques and the sequence information disclosed herein.

DNA molecules and nucleotide sequences derived from a native DNA molecule can also be defined as DNA sequences which hybridize under stringent conditions to the DNA sequences disclosed, or fragments thereof. Hybridization conditions resulting in particular degrees of stringency vary depending upon the nature of the hybridization method and the composition and length of the hybridizing DNA used. Generally, the temperature of hybridization and the ionic strength (especially the Na⁺ concentration) of the hybridization buffer determines hybridization stringency. Calculations regarding hybridization conditions required for attaining particular amounts of stringency are discussed by Sambrook et al. (Molecular Cloning: A Laboratory Manual, Cold Spring Harbor, New York, 1989, Chapters 9 and 11), herein incorporated by reference. Hybridization with a target probe labeled with [³²P]-dCTP is generally carried out in a solution of high ionic strength such as 6×SSC at a temperature that is about 5-25° C. below the melting temperature, Tₘ. An example of stringent conditions is a salt concentration of at least about 0.01 to 1.0 M Na ion concentration (or other salts) at pH 7.0 to 8.3 and a temperature of at least about 30° C. for short probes (e.g. 10 to 50 nucleotides). Stringent conditions can also be achieved with the addition of destabilizing agents such as formamide. For example, conditions of 5×SSPE (750 mM NaCl, 50 mM Na phosphate, 5 mM EDTA, pH 7.4) at 25-30° C. are suitable for allele-specific probe hybridizations.

The degeneracy of the genetic code further widens the scope of the present disclosure as it enables major variations in the nucleotide sequence of a DNA molecule while maintaining the amino acid sequence of the encoded protein. For example, the amino acid Ala is encoded by the nucleotide codon triplet GCT, GCG, GCC and GCA. Thus, the nucleotide sequence could be changed without affecting the amino acid composition of the encoded protein or the characteristics of the protein. Based upon the degeneracy of the genetic code, variant DNA molecules may be derived from a cDNA molecule using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are also comprehended by this disclosure.

Macromolecules that alter BRCA1 phorphorylation by Cds1, including variants, fragments, fusions, and polymorphisms thereof, will retain the ability to alter BRCA1 phorphorylation by Cds1, as determined using the assays disclosed herein, for example by performing an phosphorylation assay (see EXAMPLES 1-5) or a co-immunoprecipitation assay (see EXAMPLE 7). Variants and fragments of a protein may retain at least 70%, 80%, 85%, 90%, 95%, 98%, or greater sequence identity to a protein amino acid sequence and maintain the functional activity of the protein as understood by those in skilled in the art.

The simplest modifications involve the substitution of one or more amino acid residues (for example 2, 5 or 10 residues) for amino acid residues having similar biochemical properties. These so-called conservative substitutions are likely to have minimal impact on the activity of the resultant protein. Substitutional variants are those in which at least one residue in the amino acid sequence has been removed and a different residue inserted in its place. Such substitutions generally are conservative when it is desired to finely modulate the characteristics of the protein. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Substantial changes in function or immunological identity are made by selecting substitutions that are less conservative than those listed above, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

### EXAMPLE 16

### Production and Use of Antibodies

Monoclonal or polyclonal antibodies can be produced to a BRCA1 protein or functional fragments, fusions, and variants of this protein. For example, such antibodies can be produced to a BRCA1 protein having a phosphorylated serine at position 988, or substitutions at position 988, such alanine or glutamic acid.

In one embodiment, antibodies raised against BRCA1 specifically detect BRCA1 Such antibodies recognize and bind a BRCA1 protein and do not substantially recognize or bind to other proteins found in cells. Similarly, antibodies raised against BRCA1 phosphorylated at Serine 988 specifically detect the phosphorylated form, and not BRCA1 not phosphorylated at Serine 988; and antibodies raised against BRCA1 mutants having amino acid substitutions at position 988 recognize BRCA1 mutants having that particular amino acid substitution at position 988.

The determination that an antibody specifically detects the BRCA1 protein, or functional variant or fragment is made by any one of a number of standard immunoassay methods; for instance, the Western blotting technique (Sambrook et al., In Molecular Cloning: A Laboratory Manual, CSHL, New York, 1989; Scully et al., Cell 90:425-36, 1997). To determine that a given antibody preparation (such as one produced in a mouse) specifically detects a BRCA1 protein by Western blotting, total cellular protein is extracted from cells (for example, MCF-7 cells) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase. Application of an alkaline phosphatase substrate 5-bromo-4-chloro-3-indolyl phosphate/nitro blue tetrazolium results in the production of a dense blue compound by immunolocalized alkaline phosphatase. Antibodies that specifically detect a BRCA1 protein will, by this technique, be shown to bind to the BRCA1 protein band (which will be localized at a given position on the gel determined by its molecular weight). Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody-BRCA1 protein binding.

A substantially pure BRCA1 protein suitable for use as an immunogen is isolated from the transfected or transformed cells as described in U.S. Pat. No. 5,709,999. Alternatively, BRCA1 proteins and fragments, either native, or fused to an epitope tag, may be expressed and partially purified as described by Scully et al. (Science 272:123-6, 1996). Similarly, any nucleic acid sequence disclosed herein which express BRCA1, or a functional fragment or variant thereof, can be used to transform an appropriate cell type and express a BRCA1 polypeptide. For example, GST-BRCA1.4, GST-BRCA1.4 (S988A), and GST1-BRCA1.4 (S988E) expression vectors (Examples 2-4, above) can be used to express functional fragments of BRCA1 for use as an immunogen. Concentration of protein in the final preparation is adjusted, for example, by concentration on an Amicon filter device, to the level of a few micrograms per milliliter. Monoclonal or polyclonal antibody to the protein can then be prepared as follows.

Monoclonal Antibody Production by Hybridoma Fusion

Monoclonal antibody to epitopes of a BRCA1 protein, or functional fragments or variants thereof, can identified and isolated as described can be prepared from murine hybridomas according to the method of Kohler and Milstein (Nature 256:495, 1975) or derivative methods thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess un-fused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol. 70:419, 1980), and derivative methods thereof. Selected positive clones can be expanded and their monoclonal antibody product harvested for use. Detailed procedures for monoclonal antibody production are described in Harlow and Lane (Antibodies, A Laboratory Manual, CSHL, New York, 1988).

Polyclonal Antibody Production by Immunization

Polyclonal antiserum containing antibodies to heterogenous epitopes of a single protein can be prepared by immunizing suitable animals with the expressed protein which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with either inadequate or excessive doses of antigen resulting in low titer antisera. Small doses (ng level) of antigen administered at multiple intradermal sites appear to be most reliable. An effective immunization protocol for rabbits can be found in Vaitukaitis et al. (J. Clin. Endocrinol. Metab. 33:988-91, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In Handbook of Experimental Immunology, Wier, D. (ed.) chapter 19. Blackwell, 1973). Plateau concentration of antibody is usually in the range of about 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

Antibodies Raised Against Synthetic Peptides

A third approach to raising antibodies against a BRCA1 proteins and functional fragments and variants thereof, is to use synthetic peptides synthesized on a commercially available peptide synthesizer based upon the amino acid sequence of a BRCA1 protein.

By way of example only, polyclonal antibodies to specific peptides within BRCA1, were generated through well-known techniques by injecting rabbits with chemically synthesized peptide. The antibody preparations generated (GN-1385, GN-1386, and GN-1388) can be used in immunolocalization studies of the Z47 protein.

Antibodies Raised by Injection of a BRCA1 Encoding Sequence

Antibodies can be raised against a BRCA1 protein or functional fragment or variant, by subcutaneous injection of a DNA expression vector that expresses a BRCA1 protein or functional fragment or variant into laboratory animals, such as mice. Appropriate expression vectors are described herein, the references described herein, and in U.S. Pat. No. 5,709,999. Delivery of the recombinant vector into the animals may be achieved using a hand-held form of the Biolistic system (Sanford et al., Particulate Sci. Technol. 5:27-37, 1987) as described by Tang et al. (Nature 356:152-4, 1992).

Antibody preparations prepared according to these protocols are useful in quantitative immunoassays which determine concentrations of antigen-bearing substances in biological samples; they are also used semi-quantitatively or qualitatively to identify the presence of antigen in a biological sample.

Uses for Antibodies

Antibodies that recognize BRCA1 can be used to detect exposure to genotoxic stress, for example by determining whether S988 of BRCA1 in a cell is phosphorylated.

### EXAMPLE 17

Recombinant Expression of Proteins

With publicly available cDNA and corresponding amino acid sequences, as well as the disclosure herein of variants, fragments and fusions thereof, the expression and purification of any publicly known protein by standard laboratory techniques is enabled. The purified protein can be used for patient therapy. One skilled in the art will understand that a biological macromolecule that alters phosphorylation of BRCA1 by Cds to modify sensitivity to genotoxic stress, such as a protein, can be produced in any cell or organism of interest, and purified prior to administration to a subject, as an alternative to feeding the subject milk containing the recombinant clotting factor.

Methods for producing recombinant proteins are well known in the art. Therefore, the scope of this disclosure includes recombinant expression of any antigen/protein. For example, see U.S. Pat. No. 5,342,764 to Johnson et al.; U.S. Pat. No. 5,846,819 to Pausch et al.; U.S. Pat. No. 5,876,969 to Fleer et al. and Sambrook et al. (Molecular Cloning: A Laboratory Manual, Cold Spring Harbor, New York, 1989, Ch. 17, herein incorporated by reference).

For example, partial or full-length cDNA sequences, which encode for a protein (or fragment or fusion thereof), can be ligated into bacterial expression vectors. Methods and plasmid vectors for producing fusion proteins and intact native proteins (or variants or fragments thereof) in bacteria are described in Sambrook et al. (Sambrook et al., In Molecular Cloning: A Laboratory Manual, Ch. 17, CSHL, New York, 1989). Vector systems suitable for the expression of lacZ fusion genes include the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986).

A nucleic acid sequence can also be transferred from its existing context to other cloning vehicles, such as other plasmids, bacteriophages, cosmids, animal viruses and yeast artificial chromosomes (YACs) (Burke et al., Science 236:806-12, 1987). These vectors may then be introduced into a variety of hosts including somatic cells, and simple or complex organisms, such as bacteria, fungi (Timberlake and Marshall, Science 244:1313-7, 1989), invertebrates, plants (Gasser and Fraley, Science 244:1293, 1989), and animals (Pursel et al., Science 244:1281-8, 1989), which cell or organisms are rendered transgenic by the introduction of a heterologous cDNA.

The transfer of DNA into eukaryotic, such as human or other mammalian cells, is a conventional technique. The vectors are introduced into the recipient cells as pure DNA (transfection) by, for example, precipitation with calcium phosphate (Graham and vander Eb, Virology 52:466, 1973) or strontium phosphate. (Brash et al., Mol. Cell Biol. 7:2013, 1987), electroporation (Neumann et al., EMBO J 1:841, 1982), lipofection (Felgner et al., Proc. Natl. Acad. Sci. USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors, for example, retroviruses (Bernstein et al., Gen. Engr'g 7:235, 1985), adenoviruses (Ahmad et al., J. Virol. 57:267, 1986), or Herpes (Spaete et al., Cell 30:295, 1982).

### EXAMPLE 18

### Peptide Modifications

Proteins that alter phosphorylation of BRCA1 by Cds to modify sensitivity to genotoxic stress, can be modified using a variety of chemical techniques to produce derivatives having essentially the same activity as the unmodified peptides, and optionally having other desirable properties. For example, carboxylic acid groups of the peptide, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6- membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chain can be converted to C₁-C₁₆ alkoxy or to a C₁-C₁₆ ester using well-recognized techniques. Phenyl and phenolic rings of the peptide side chain can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides disclosed herein to select and provide conformational constraints to the structure that result in enhanced stability. For example, a carboxyl-terminal or amino-terminal cysteine residue can be added to the peptide, so that when oxidized the peptide will contain a disulfide bond, generating a cyclic peptide. Other peptide cyclizing methods include the formation of thioethers and carboxyl- and amino-terminal amides and esters.

To maintain a functional peptide, particular peptide variants will differ by only a small number of amino acids from the peptides disclosed herein. Such variants can have deletions (for example of 1-3 or more amino acids), insertions (for example of 1-3 or more residues), or substitutions that do not interfere with the desired activity of the peptides. Substitutional variants are those in which at least one residue in the amino acid sequence has been removed and a different residue inserted in its place. In particular embodiments, such variants have amino acid substitutions of single residues, for example 1, 3, 5 or even 10 substitutions in a protein.

Peptidomimetic and organomimetic embodiments are also disclosed herein, whereby the three-dimensional arrangement of the chemical constituents of such peptido- and organomimetics mimic the three-dimensional arrangement of the peptide backbone and component amino acid sidechains in the peptide, resulting in such peptido- and organomimetics of a protein that alters phosphorylation of BRCA1 by Cds1 to modify sensitivity to genotoxic stress. For computer modeling applications, a pharmacophore is an idealized, three-dimensional definition of the structural requirements for biological activity. Peptido- and organomnimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in CADD.

### EXAMPLE 19

### Peptide Synthesis and Purification

Proteins that alter phosphorylation of BRCA1 by Cds to modify sensitivity to genotoxic stress, and variants, fusions, polymorphisms, fragments, and mutants thereof, can be chemically synthesized by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### EXAMPLE 20

### In vivo Expression

The present disclosure provides methods of expressing a protein that alters phosphorylation of BRCA1 by Cds in a cell or tissue in vivo, to modify sensitivity to genotoxic stress. In one embodiment, transfection of the cell or tissue occurs in vitro. In this example, the cell or tissue is removed from a subject and then transfected with an expression vector containing cDNA. The transfected cells will produce functional protein and can be reintroduced into the subject. In another embodiment, a nucleic acid is administered to the subject directly, and transfection occurs in vivo.

Retroviruses have a high efficiency of infection and stable integration and expression (Orkin et al., Prog. Med. Genet. 7:130-142, 1988). For example, a full-length BRCA1 gene or cDNA encoding a mutation at amino acid residue 988 can be cloned into a retroviral vector and driven from either its endogenous promoter, a heterologous promoter (constituitive or inducible) or from the retroviral LTR (long terminal repeat). Other viral transfection systems may also be utilized for this type of approach, including adenovirus, adeno-associated virus (AAV) (McLaughlin et al., J. Virol. 62:1963-1973, 1988), Vaccinia virus (Moss et al., Annu. Rev. Immunol. 5:305-324, 1987), Bovine Papilloma virus (Rasmussen et al., Methods Enzymol. 139:642-654, 1987), members of the herpesvirus group such as Epstein-Barr virus (Margolskee et al., Mol. Cell. Biol. 8:2837-2847, 1988), or lentivirus and related vectors (U.S. Pat. No. 6,013,516).

Very large nucleic acid inserts can be integrated into viral systems. Kochanek et al. (Proc. Natl. Acad. Sci. USA 93:5731-9, 1996) demonstrated efficient packaging in an adenoviral system of a 28.2 kb expression cassette for use in gene transfer therapy. Parks and Graham demonstrated packaging of vectors with sizes ranging from 15.1 to 33.6 kb (Proc. Natl. Acad. Sci. USA 93:13565-70, 1996; J. Virol. 71:3293-8, 1997).

Recent developments in in vivo expression techniques include the use of RNA-DNA hybrid oligonucleotides, as described by Cole-Strauss, et al. (Science 273:1386-9, 1996). This technique may allow for site-specific integration of cloned sequences, thereby permitting accurately targeted gene modification.

It is possible to use non-infectious methods of delivery. For instance, lipidic and liposome-mediated gene delivery has recently been used successfully for transfection with various genes (see Templeton and Lasic, Mol. Biotechnol. 11: 175-80, 1999; Lee and Huang, Crit. Rev. Ther. Drug Carrier Syst. 14:173-206; and Cooper, Semin. Oncol. 23:172-87, 1996), and for delivery of peptides. For instance, cationic liposomes have been analyzed for their ability to transfect monocytic leukemia cells, and shown to be a viable alternative to using viral vectors (de Lima et al., Mol. Membr. Biol. 16:103-9, 1999). Such cationic liposomes can also be targeted to specific cells through the inclusion of, for instance, monoclonal antibodies or other appropriate targeting ligands (Kao et al., Cancer Gene Ther. 3:250-6, 1996).

Delivery of the polypeptides disclosed herein to tumor cells enhances the sensitivity of such tumor cells to genotoxic agents such as ionizing radiation, ultraviolet radiation, and chemotherapeutic agents (such as all those described in Slapak and Kufe, Principles of Cancer Therapy, Ch. 86 in Harrison's Principles of Internal Medicine, 14^{th} Ed. 1998).

For example, delivery of a vector expressing a BRCA1 polypeptide, or functional fragment or variant thereof, where the BRCA1 polypeptide includes an S988 mutation (for example, S988A), will enhance the sensitivity of a cancerous tumor in a subject to genotoxic agents. For enhancing sensitivity of tumor cells to genotoxic agents, the S988 mutation is of a type that reduces the ability of the cell to repair damage after exposure to genotoxic stress. Such amino acid substitutions at position 988 would include, for example, ala, arg, asn, cys, gln, gly, his, ile, leu, lys, met, phe, thr, trp, tyr, and val. These amino acid substitutions are made by standard mutagenesis techniques, and were used to generate the various S988A and S988E mutations described in the Examples herein. The same techniques may be used to create any amino acid substitution at position 988. Mutations in a nucleic acid sequence which leads to these amino acid substitutions can be generated in a nucleic acid expressing full-length BRCA1 protein, or alternatively in a functional fragment or variant thereof. Each amino acid substitution is tested for reduced ability to disperse from nuclear foci according the methods described in Example 6, impaired enhancement of p21 -mediated expression following irradiation according to Example 9, and/or persistent complex formation with CtIP following irradiation according to Example 10. BRCA1 variants that exhibit one or more of these characteristics will, upon delivery to tumor cells, enhance their sensitivity to genotoxic agents.

For example, a vector expressing a functional fragment of BRCA1 comprising at least 70% homology to amino acid residues 758-1064 of human BRCA1, wherein ala is substituted for serine at position 988, is administered to a patient with skin disease. The disease may be a skin tumor, for example, melanoma, squamous cell carcinoma or basal cell carcinoma (Sober et al., Melanoma and Other Skin Cancers, Ch. 88 in Harrison's Principles of Internal Medicine, Fauci et al., eds., 14th edition 1998). The disease may also be, for example, a hyperprolifeative lesion or a premalignant lesion. Such diseases include psoriasis, vitiligo, atopic dermatitis, or hyperproliferative or UV-induced dermatoses. The vector is administered to the skin of the subject at or near the site of the lesion, using any of the vectors and techniques described in this application and references herein. The subject is then treated with ionizing radiation in accordance with standard protocols known in the radiation therapy art (see Gunderson et al., eds., Clinical Radiation Oncology, 1st ed 2000). Alternatively, a subject with a hyperproliferative lesion such as psoriasis may be treated with UV radiation, with or without additional adjunctive therapy (Swerlick et al, Eczema, Psoriasis, Cutaneous Infections, Acne, and Other Common Skin Disorders, Chapter 55 in Harrison's Principles of Internal Medicine, Fauci et al., eds., 14th edition 1998; and Arndt et al. eds., Cutaneous Medicine and Surgery: An Integrated Program, 1 st. ed. 1996).

Peptides which have BRCA1 activity and carry a mutation at the position corresponding to S988 can be supplied to tumor cells to enhance their sensitivity to genotoxic stress. Protein can be produced by expression of the cDNA sequence in bacteria, for example, using known expression vectors. For example, the expression vector GST-BRCA1.4 (S988A) is used to express and purify an S988A functional fragment of BRCA1 for peptide therapy. In addition, the techniques of synthetic chemistry can be employed to synthesize BRCA1 protein having an amino acid substitution at the position corresponding to BRCA1 S988, for example a peptide of the sequence CRIPPLFPIKAFVKTK (SEQ ID NO:4) can be synthesized and used to practice the methods disclosed herein (sequence corresponds to residues 978-993 of human BRCA1, with ala substituted for ser at residue 988). The preparation is substantially free of other human proteins. This can be accomplished by synthesis in a microorganism or in vitro, using methods disclosed herein, such as EXAMPLES 17 and 19.

Such peptides can be introduced into cells by microinjection or by use of liposomes, for example. Alternatively, some active molecules may be taken up by cells, actively or by diffusion. The peptides will enhance sensitivity to genotoxic agents, for example by enhancing sensitivity to UV or ionizing radiation after application to treat skin diseases.

Also by way of example, BRCA1 S988 mutants which inhibit BRCA1-mediated pathways (such as those described above in this Example), may be used as therapy for breast cancer. For example, such mutants may be delivered by gene or peptide therapy to a breast tumor as an adjunct to breast radiotherapy, with or without lumpectomy. Such mutants enhance sensitivity of breast tumor cells to ionizing radiation and/or chemotherapy agents.

Similarly, expression of a kinase-dead hCds1 mutant, or similar kinase-dead or kinase-impaired mutant or any homolog or functional fragment or variant of Cds1, will enhance sensitivity to genotoxic agents. For example, a nucleic acid encoding hCds1 [K249R] mutant described in Example 6, or functional fragment or variant thereof, may be transferred to an appropriate gene therapy vector as described in this Example (or used directly as the nucleic acid vector described in Example 6, without further manipulation), and delivered to tumor cells in the same manner as described for BRCA1 S988 mutants. Such hCds1 mutants enhance sensitivity to genotoxic agents. Without intending to be bound by any particular mechanism or explanation, it is currently believed that such kinase-dead or kinase-impaired hCds1 mutants enhance sensitivity to genotoxic agents by inhibiting DNA damage repair, such as that mediated by BRCA1.

The methods disclosed herein can also be used to reduce sensitivity to genotoxic agents. Some BRCA1 S988 mutants have been shown by the examples herein to increase BRCA1-dependent signaling pathways. These pathways are typically activated in response to genotoxic stimuli such as ionizing radiation. For example, S988E mutants enhance expression from the p21waf1/cip1 promoter (Example 9), and thereby enhance cell cycle checkpoint regulation at the G1-S boundary. Thus, expression of BRCA1 S988E mutants, or fragment or variant thereof, enhances cell survival after exposure to genotoxic stress by allowing greater opportunity for cellular repair prior to entry into S phase. Using the vectors and methods described in this Example, such mutants could be delivered to normal tissues in a subject undergoing genotoxic therapy, to reduce the impact of such therapy. For example, a gene therapy vector expressing a S988E mutant (or BRCA1 polypeptide fragment containing a S988E mutation) can be applied to normal skin surrounding a psoriatic lesion prior to treating the lesion with psoralens and UV irradiation. In another example, such a vector or peptide could be applied to the skin of a subject undergoing radiotherapy to prevent radiation-induced skin injury. In yet another example, a vector encoding an S988E mutant operably linked to a cardiac-specific promoter could be delivered by gene therapy to reduce cardiac injury caused by chemotherapy agents such as anthracyclines.

### EXAMPLE 21

### Viral Vectors for in vivo Gene Expression

Adenoviral vectors include essentially the complete adenoviral genome (Shenk et al., Curr. Top. Microbiol. Immunol. 111:1-39, 1984). Alternatively, the adenoviral vector is a modified adenoviral vector in which at least a portion of the adenoviral genome has been deleted. In one embodiment, the vector includes an adenoviral 5′ ITR; an adenoviral 3′ ITR; an adenoviral encapsidation signal; a DNA sequence encoding a therapeutic agent; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. In another embodiment, the vector is an adeno-associated virus (AAV) such as described in U.S. Pat. No. 4,797,368 (Carter et al.) and in McLaughlin et al. (J. Virol. 62:1963-73, 1988) and AAV type 4 (Chiorini et al. J. Virol. 71:6823-33, 1997) and AAV type 5 (Chiorini et al. J. Virol. 73:1309-19, 1999)

Such a vector can be constructed according to standard techniques, using a shuttle plasmid which contains, beginning at the 5′ end, an adenoviral 5′ ITR, an adenoviral encapsidation signal, and an E1a enhancer sequence; a promoter (which may be an adenoviral promoter or a foreign promoter); a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid can also include a selectable marker and an origin of replication. The origin of replication may be a bacterial origin of replication. A desired DNA sequence encoding a therapeutic agent can be inserted into the multiple cloning site of the plasmid.

The plasmid can be used to produce an adenoviral vector by homologous recombination with a modified or mutated adenovirus in which at least the majority of the E1 and E3 adenoviral DNA sequences have been deleted. Homologous recombination can be effected through co-transfection of the plasmid vector and the modified adenovirus into a helper cell line, such as 293 cells, by CaPO₄ precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one embodiment, the adenovirus is constructed by using a yeast artificial chromosome (or YAC) containing an adenoviral genome according to the method described in Ketner et al. (Proc. Natl. Acad. Sci. USA, 91:6186-90, 1994), in conjunction with the teachings contained herein. In this embodiment, the adenovirus YAC is produced by homologous recombination in vivo between adenoviral DNA and YAC plasmid vectors carrying segments of the adenoviral left and right genomic termini. A DNA sequence encoding a therapeutic agent then is cloned into the adenoviral DNA. The modified adenoviral genome then is excised from the adenovirus YAC to be used to generate adenoviral vector particles as herein described.

Adenoviral particles are administered in an amount effective to produce a therapeutic effect in a subject. The exact dosage of adenoviral particles to be administered is dependent upon a variety of factors, including the age, weight, and sex of the subject to be treated, and the nature and extent of the disease or disorder to be treated. The adenoviral particles may be administered as part of a preparation having a titer of adenoviral particles of at least 1×10¹⁰ pfu/ml, and in general not exceeding 2×10¹¹ pfu/ml. The adenoviral particles can be administered in combination with a pharmaceutically acceptable carrier in a volume up to 10 ml. The pharmaceutically acceptable carrier may be, for example, a liquid carrier such as a saline solution, protamine sulfate (Elkins-Sinn, Inc., Cherry Hill, N.J.), or Polybrene (Sigma Chemical) as well as those described in EXAMPLE 22.

In another embodiment, the viral vector is a retroviral vector. Retroviruses can be used for in vivo gene expression because they have a high efficiency of infection and stable integration and expression (Orkin et al., 1988, Prog. Med. Genet. 7:13042). A full length Edaradd gene or cDNA can be cloned into a retroviral vector and driven from either its endogenous promoter or from the retroviral LTR. Examples of retroviral vectors which can be used include, but are not limited to, MMLV, spleen necrosis virus, and vectors derived from retroviruses such as RSV, Harvey Sarcoma Virus, avian leukosis virus, HIV, myeloproliferative sarcoma virus, and mammary tumor virus. The vector is generally a replication defective retrovirus particle.

Retroviral vectors are useful to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. Examples include digestion with the appropriate restriction endonuclease or, in some instances, with Bal 31 exonuclease to generate fragments containing appropriate portions of the packaging signal.

Other viral transfection systems may also be utilized for this type of approach, including Vaccinia virus (Moss et al., 1987, Annu. Rev. Immunol. 5:305-24), Bovine Papilloma virus (Rasmussen et al., 1987, Methods Enzymol. 139:642-54) or members of the herpes virus group such as Epstein-Barr virus (Margolskee et al., 1988, Mol. Cell. Biol. 8:2837-47). In another embodiment RNA-DNA hybrid oligonucleotides, as described by Cole-Strauss et al. (Science 273:1386-9, 1996) are used. This technique can allow for site-specific integration of cloned sequences, permitting accurately targeted gene replacement.

New genes can be incorporated into proviral backbones in several ways. In the most straightforward constructions, the structural genes of the retrovirus are replaced by a single gene which then is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

### EXAMPLE 22

### Pharmaceutical Compositions and Modes of Administration

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal, vaginal and intestinal mucosa, etc.) and may be administered together with other biologically active agents. Administration can be systemic or local. Pharmaceutical compositions can be introduced into the central nervous system by any suitable route, including intraventricular and intrathecal injection; intraventricular injection may be facilitated by an intraventricular catheter, for example, attached to a reservoir, such as an Ommaya reservoir.

In one embodiment, pharmaceutical compositions disclosed herein are delivered locally to the area in need of treatment, for example, by local infusion during surgery, topical application, e.g., in conjunction with a wound dressing after surgery, by injection, through a catheter, by a suppository or an implant, such as a porous, non-porous, or gelatinous material, including membranes, such as silastic membranes, or fibers. In one embodiment, administration can be by direct administration at a site where hair growth, tooth growth, epithelial, or sweat gland growth is desired.

The use of liposomes as a delivery vehicle is one delivery method of interest. [symbol] The liposomes fuse with the target site and deliver the contents of the lumen intracellularly. The liposomes are maintained in contact with the target cells for a sufficient time for fusion to occur, using various means to maintain contact, such as isolation and binding agents. Liposomes can be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 1991, 266:3361) can be used.

The present disclosure also provides pharmaceutical compositions which include a therapeutically effective amount of a biological macromolecule that alters phosphorylation of BRCA1 by Cds1, alone or with a pharmaceutically acceptable carrier. Furthermore, the pharmaceutical compositions or methods of treatment can be administered in combination with other therapeutic treatments, such as chemotherapeutic agents.

Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of a biological macromolecule that alters phosphorylation of BRCA1 by Cds1, such as a nucleic acid or protein herein disclosed. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (e.g., powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure comprising medicaments can be prepared with conventional pharmaceutically acceptable carriers, adjuvants and counterions as would be known to those of skill in the art.

The amount of a biological macromolecule that alters phosphorylation of BRCA1 by Cds1 effective in the treatment of a particular disorder or condition will depend on the nature of the disorder or condition, and can be determined by standard clinical techniques. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

The disclosure provides compositions including one or more biological macromolecules that alter phosphorylation of BRCA1 by Cds1, for example a composition that is comprised of at least 50%, for example at least 90%, of a biological macromolecule in the composition. Such compositions are useful as therapeutic agents when constituted as pharmaceutical compositions with the appropriate carriers or diluents.

Administration of Nucleic Acid Molecules

In an embodiment in which a biological macromolecule that alters phosphorylation of BRCA1 by Cds1 is a nucleic acid is employed to allow expression of the nucleic acid in a cell, the nucleic acid is delivered intracellularly (e.g., by expression from a nucleic acid vector or by receptor-mediated mechanisms). In an embodiment where the therapeutic molecule is a nucleic acid administration can be achieved by an appropriate nucleic acid expression vector which is administered so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 1991, 88:1864-8), etc. Alternatively, the nucleic acid can be introduced intracellularly and incorporated within host cell DNA for expression, by homologous recombination.

The vector pcDNA, is an example of a method of introducing the foreign cDNA into a cell under the control of a strong viral promoter (CMV) to drive the expression. However, other vectors can be used (see EXAMPLES 17 and 21). Other retroviral vectors (such as pRETRO-ON, Clontech), also use this promoter but have the advantages of entering cells without any transfection aid, integrating into the genome of target cells only when the target cell is dividing (as cancer cells do, especially during first remissions after chemotherapy) and they are regulated. It is also possible to turn on the expression of a nucleic acid by administering tetracycline when these plasmids are used. Hence these plasmids can be allowed to transfect the cells, then administer a course of tetracycline with a course of chemotherapy to achieve better cytotoxicity.

Other plasmid vectors, such as pMAM-neo (Clontech) or pMSG (Pharmacia) use the MMTV-LTR promoter (which can be regulated with steroids) or the SV10 late promoter (pSVL, Pharmacia) or metallothionein-responsive promoter (pBPV, Pharmacia) and other viral vectors, including retroviruses. Examples of other viral vectors include adenovirus, AAV (adeno-associated virus), recombinant HSV, poxviruses (vaccinia) and recombinant lentivirus (such as HIV). These vectors achieve the basic goal of delivering into the target cell the cDNA sequence and control elements needed for transcription. The present disclosure includes all forms of nucleic acid delivery, including synthetic oligos, naked DNA, plasmid and viral, integrated into the genome or not.

Administration of Antibodies

In an embodiment where the therapeutic molecule is a specific-binding agent, such as an antibody that recognizes a protein, for example a BRCA1 protein, administration can be achieved by direct injection, or by use of microparticle bombardment (e.g., a gene gun; Biolistic, Dupont), or coating with lipids or cell-surface receptors or transfecting agents. Similar methods can be used to administer a protein, or fragments, fusions, or variants thereof.

Having illustrated and described the principles of hCds1, its interaction with BRCA1, and the role of BRCA1 S988 in gene expression, DNA repair and cell survival following genotoxic stress, it will be apparent to one skilled in the art that the disclosure can be modified in arrangement and detail without departing from such principles. I therefore claim as my invention all that comes within the scope and spirit of these claims.

1-56. (Canceled)

57. A BRCA1 peptide-specific antibody, wherein the BRCA1 peptide comprises a phosphorylated serine residue at a position corresponding to amino acid residue 988 of a human BRCA1 polypeptide.

58. The BRCA1 peptide-specific antibody of claim 57, wherein the antibody is polyclonal.

59. The BRCA1 peptide-specific antibody of claim 57, wherein the antibody is monoclonal.

60. A BRCA1 peptide-specific antibody, wherein the BRCA1 peptide comprises an amino acid residue other than serine at a position corresponding to 988 of a human BRCA1 polypeptide.

61. The BRCA1 peptide-specific antibody of claim 58, wherein the amino acid residue other than serine is alanine or glutamic acid.

62-63. (Canceled)


SEQUENCE LISTING (4 sequences)

SEQ ID NO: 1 - protein, 16 aa, Homo sapiens
CRIPPLFPIKSFVKTK

SEQ ID NO: 2 - DNA, 19 bp, Homo sapiens
agcttggtcactgtgaccg

SEQ ID NO: 3 - DNA, 19 bp, Homo sapiens
catccggtcacagtgacca

SEQ ID NO: 4 - protein, 16 aa, Homo sapiens
CRIPPLFPIKAFVKTK